(19) United States
(12) Patent Application Publication
(10) Pub. No.: US 2017/0130202 A1
(43) Pub. Date: May 11, 2017

(54) METHODS RESPECTIVELY FOR PRODUCING MESODERMAL CELLS AND HEMATOPOIETIC CELLS

(71) Applicant: KYOTO UNIVERSITY, Kyoto-shi, Kyoto (JP)

(72) Inventors: Tatsutoshi Nakahata, Kyoto-shi, Kyoto (JP); Megumu Saito, Kyoto-shi, Kyoto (JP); Akira Niwa, Kyoto-shi, Kyoto (JP); Yoshinori Sugimine, Kyoto-shi, Kyoto (JP)

(21) Appl. No.: 15/320,722

(22) PCT Filed: Jun. 24, 2015
(86) PCT No.: PCT/JP2015/068191

§ 371 Date: Dec. 20, 2016

(30) Foreign Application Priority Data
Jun. 24, 2014 (JP) 2014-128988

(51) Int. Cl. C12N 5/077; A61L 27/38; C12N 5/0786; C12N 5/078; C12N 5/0735; A61K 35/28

(52) U.S. Cl. CPC C12N 5/0652; C12N 5/0606; A61K 35/28; C12N 5/0645; C12N 5/0641; A61L 27/3834; A61L 27/3895; C12N 2506/45; C12N 2513/00; C12N 2533/54; C12N 2533/30; C12N 2501/155; C12N 2501/165; C12N 2501/115; C12N 2501/125; C12N 2506/02; C12N 2501/2303; C12N 2501/26; C12N 2501/145; C12N 2501/22; C12N 2501/14; A61K 2035/124

(57) ABSTRACT

The present invention aims to provide a novel method for producing mesodermal cells, and a graft material containing mesodermal cells obtained by this method. The present invention also aims to provide a novel method for producing hematopoietic cells, and a therapeutic agent for blood diseases containing a hematopoietic cell obtained by the method. These objects can be achieved by providing a novel method for producing mesodermal cells and/or hematopoietic cells, which method includes culturing pluripotent stem cells in contact with a three-dimensional support.

### TECHNICAL FIELD

The present invention relates to a novel method for producing mesodermal cells, mesodermal cells supported by a three-dimensional support obtained by this method, and a graft material containing the mesodermal cells supported by a three-dimensional support; and a culture vessel retaining a plurality of three-dimensional supports supporting mesodermal cells. The present invention also relates to a novel method for producing hematopoietic cells, and a therapeutic agent for blood diseases containing a hematopoietic cell obtained by this method.

### BACKGROUND ART

In treatment of blood-related diseases represented by leukemia, and in surgical treatment, it is very important to stably amplify and supply blood cells in an amount necessary for the treatment. For this purpose, a number of medical workers have devised various means for securing the blood cells. For example, collection of blood from donors and induction of differentiation from cord blood or bone marrow cells have been conventionally carried out.

In recent years, attempts are being made to efficiently amplify, for example, hematopoietic stem cells or hematopoietic progenitor cells, which are sources for production of blood cells, or erythrocytes or neutrophils, which are more matured cells, using cells having pluripotency such as embryonic stem cells (ES cells) or induced pluripotent stem (iPS) cells obtained by introduction of undifferentiated-cell-specific genes into somatic cells (e.g., Patent Documents 1 and 2).

Examples of methods for inducing differentiation of ES cells or iPS cells into hematopoietic stem cells or hematopoietic progenitor cells that have been reported so far include methods by formation of embryoid bodies and addition of cytokines (Non-patent Documents 1 to 3), a method by co-culture with stromal cells derived from a different species (Non-patent Document 4), and a method using a serum-free medium (Patent Document 3). Similarly, methods for inducing differentiation into erythrocytes have been reported by, for example, Non-patent Documents 1 to 4 and Patent Document 3, and methods for inducing differentiation into neutrophils have been reported by, for example, Non-patent Documents 3 and 5.

However, further improvement of methods for induction of differentiation into blood cells is necessary for enabling stable supply of a large amount of blood cells. Thus, development of a novel technique suitable for application to medicine has been demanded.

### PRIOR ART DOCUMENTS

### Patent Documents

[Patent Document 1] U.S. Pat. No. 5,843,780
[Patent Document 2] WO 2007/069666
[Patent Document 3] WO 2011/115308

### Non-Patent Documents

Non-patent Document 1: Chadwick et al. Blood 2003, 102: 906-15
Non-patent Document 2: Vijayaragavan et al. Cell Stem Cell 2009, 4: 248-62
Non-patent Document 3: Saeki et al. Stem Cells 2009, 27: 59-67
Non-patent Document 4: Niwa A et al. J Cell Physiol. 2009 November; 221(2): 367-77
Non-patent Document 5: Morishima et al. J Cell Physiol. 2011 May; 226(5): 1283-91

### SUMMARY OF THE INVENTION

### Problems to be Solved by the Invention

An object of the present invention is to provide a novel method for producing mesodermal cells, mesodermal cells supported by a three-dimensional support obtained by this method, and a graft material containing the mesodermal cells supported by a three-dimensional support; and a culture vessel retaining a plurality of three-dimensional supports supporting mesodermal cells. Another object of the present invention is to provide a novel method for producing hematopoietic cells, and a therapeutic agent for blood diseases containing a hematopoietic cell obtained by this method.

### Means for Solving the Problems

The present inventors intensively studied to solve the above problems, and, as a result, succeeded in differentiation induction of pluripotent stem cells into mesodermal cells and/or hematopoietic cells by culturing the pluripotent stem cells under conditions where the pluripotent stem cells are in contact with a three-dimensional support. Similarly, the present inventors succeeded in differentiation induction of mesodermal cells into hematopoietic cells by culturing the mesodermal cells under conditions where the mesodermal cells are in contact with a three-dimensional support. Further, the present inventors discovered for the first time that hematopoietic cells can be stably supplied for a long period by culturing pluripotent stem cells and/or mesodermal cells under conditions where these cells are in contact with a three-dimensional support. The present invention was completed based on such findings.

That is, the present invention provides the following.
(1) A method for producing mesodermal cells from pluripotent stem cells, comprising culturing pluripotent stem cells in contact with a three-dimensional support to induce mesodermal cells.
(2) The method according to (1), wherein said mesodermal cells are KDR-, and CD34-positive cells.
(3) The method according to (1) or (2), wherein said three-dimensional support is a collagen sponge.
(4) The method according to (3), wherein said collagen sponge is a collagen sponge reinforced with polyethylene terephthalate fibers.
(5) The method according to any one of (1) to (4), wherein the contact between said pluripotent stem cells and said three-dimensional support occurs on a surface and/or in the inside of said three-dimensional support.
(6) The method according to any one of (1) to (5), wherein the step of culturing said pluripotent stem cells in contact with said three-dimensional support comprises:

(i) culturing said pluripotent stem cells in a medium containing BMP4; and

(ii) culturing cells obtained in Step (i) in a medium containing VEGF, bFGF, and SCF.
(7) The method according to (6), wherein the culture periods of the steps (i) and (ii) are 1 to 5 days and 0.5 to 3 days, respectively.
(8) The method according to (7), wherein the culture periods of the steps (i) and (ii) are 3 days and 1 day, respectively.
(9) The method according to any one of (6) to (8), wherein preculture is carried out by bringing pluripotent stem cells into contact with the three-dimensional support before the step (i).
(10) The method according to any one of (1) to (9), wherein said pluripotent stem cells are human iPS cells.
(11) The method according to any one of (1) to (10), wherein said pluripotent stem cells are small clusters or single cells.
(12) The method according to (11), wherein said pluripotent stem cells are single cells.
(13) A mesodermal cell supported by a three-dimensional support, produced by the method according to any one of (1) to (12).
(14) A graft material comprising the mesodermal cell supported by a three-dimensional support according to (13).
(15) A culture vessel retaining one or more three-dimensional supports, wherein mesodermal cells are supported by said three-dimensional support(s), and said mesodermal cells are cells produced by the method according to any one of (1) to (12).
(16) A method for producing hematopoietic cells, comprising:

(a) producing mesodermal cells in contact with a three-dimensional support by the method according to any one of (1) to (12); and

(b) culturing the obtained mesodermal cells retained by the three-dimensional support in a culture vessel to induce hematopoietic cells.
(17) The method according to (16), wherein said hematopoietic cells are myeloid cells, monocytic cells, or erythroid cells.
(18) The method according to (16) or (17), wherein said hematopoietic cells are myeloid cells.
(19) The method according to (16) or (17), wherein said hematopoietic cells are monocytic cells.
(20) The method according to (16) or (17), wherein said hematopoietic cells are erythroid cells.
(21) The method according to (18), wherein the step (b) comprises culturing the mesodermal cells retained by the three-dimensional support in a medium containing SCF, IL-3, Flt3L, and thrombopoietin (TPO).
(22) The method according to (19), wherein the step (b) comprises:

(i) culturing the mesodermal cells retained by the three-dimensional support in a medium containing SCF, IL-3, Flt3L, and TPO;

(ii) culturing cells obtained in the step (i) in a medium containing SCF, IL-3, Flt3L, TPO, and M-CSF; and

(iii) culturing cells obtained in the step (ii) in a medium containing Flt3L, M-CSF, and GM-CSF.
(23) The method according to (20), wherein the step (b) comprises culturing the mesodermal cells retained by the three-dimensional support in a medium containing erythropoietin (EPO) and SCF.
(24) The method according to (21) or (23), wherein the culture period of the step (b) is 16 to 41 days.
(25) The method according to (24), wherein the culture period of the step (b) is 31 days.
(26) The method according to (22), wherein the culture periods of the steps (i), (ii), and (iii) are 2 to 5 days, 2 to 5 days, and 8 to 33 days, respectively.
(27) The method according to (26), wherein the culture periods of the steps (i), (ii), and (iii) are 3 days, 3 days, and 23 days, respectively.
(28) A therapeutic agent for blood diseases, comprising a hematopoietic cell produced by the method according to any one of (16) to (27).

### Effect of the Invention

By the present invention, a large amount of hematopoietic cells can be stably supplied. The effect can be exerted more favorably in cases where the differentiation induction is carried out using pluripotent stem cells in the form of single cells. By this, treatment of various blood diseases becomes possible.

### BRIEF DESCRIPTION OF THE DRAWINGS

FIG. 1 shows the results of differentiation of small clusters of undifferentiated pluripotent stem cells (PSCs) into hematopoietic cells. Panel (a) shows a micrograph showing small clusters of PSCs on a CS. Each arrowhead indicates a small cluster of PSCs (KhES1). The scale bar represents 500 μm. Panel (b) shows the result of flow cytometry of hematopoietic progenitor cells obtained by differentiation induction from KhES1 on Day 6. In the flow cytometry analysis, the following antibodies were used: KDR (CD309)-Alexa fluor 647 (Biolegend), CD34-PE (BD Biosciences), and CD45-PE (BD Biosciences). Exclusion of dead cells and debris was carried out by DAPI staining. Panel (c) shows the ratio of KDR-, and CD34-positive hematopoietic progenitor cells on Day 6. Each error bar represents the standard deviation (S.D.). All data are representative data each of which is based on at least three independent experiments.

FIG. 2 shows the results of differentiation into particular hematopoietic cell lineages. Panels (a) and (b) show the results of differentiation into myeloid cells. Panel (a) shows the result of flow cytometry, and Panel (b) shows a photograph showing the result of Giemsa staining (May-Giemsa staining). The scale bar in Panel (b) represents 20 μm. Panels (c) and (d) show the results of differentiation into monocytic cells. Panel (c) shows the result of flow cytometry, and Panel (d) shows a photograph showing the result of Giemsa staining (May-Giemsa staining). The scale bar in Panel (d) represents 20 μm. Panels (e) and (f) show the results of differentiation into erythroid cells. Panel (e) shows the result of flow cytometry, and Panel (f) shows a photograph showing the result of Giemsa staining (May-Giemsa staining). The scale bar in Panel (f) represents 20 μm. Panel (g) shows the numbers of hematopoietic cells recovered after different culture periods. All data are representative data each of which is based on at least three independent experiments. In the flow cytometry analysis, the following antibodies were used: CD43-APC (BD Biosciences), CD45-PE (BD Biosciences), CD71-APC (BD Biosciences), CD235a-PE (BD Biosciences), and CD14-APC (Beckman Coulter). Panel (h) shows the result of flow cytometry. Panel (i) shows the result of a colony formation test. CFU-G represents the granulocyte colony-forming unit; CFU-GM represents the granulocyte macrophage-colony forming unit; and BFU-E represents the erythroblast burst-forming unit.

FIG. 3 shows the results of differentiation of single undifferentiated PSCs into hematopoietic cells. Panel (a) shows a micrograph showing a CS in the beginning of differentiation. No clear aggregate of cells was found. The scale bar represents 500 μm. Panels (b) to (e) show the result of flow cytometry of hematopoietic progenitor cells obtained by differentiation induction from KhES1 on Day 6. Exclusion of dead cells and debris was carried out by DAPI staining. Panel (b) shows the total cell number; Panel (c) shows the ratio of CD34-, and KDR-positive hematopoietic progenitor cells (HPCs); Panel (d) shows the number of CD34-, and KDR-positive HPCs; and Panel (e) shows the result of flow cytometry. Panel (f) shows the result of flow cytometry of myeloid cells on Day 38. Panel (g) shows the result of Giemsa staining of hematopoietic progenitor cells obtained by differentiation induction from KhES1 on Day 6. The scale bar represents 20 μm. Panels (h) to (j) and Panel (q) show the results of flow cytometry of hematopoietic progenitor cells obtained by differentiation induction from 201B7 on Day 6. Exclusion of dead cells and debris was carried out by DAPI staining. Panel (h) shows the total cell number; Panel (i) shows the ratio of CD34-, and KDR-positive HPCs; Panel (j) shows the number of CD34-, and KDR-positive HPCs; and Panel (q) shows the result of flow cytometry. Panels (k) to (m) and Panel (r) show the results of flow cytometry of hematopoietic progenitor cells obtained by differentiation induction from 402B2 on Day 6. Exclusion of dead cells and debris was carried out by DAPI staining. Panel (k) shows the total cell number; Panel (l) shows the ratio of CD34-, and KDR-positive HPCs; Panel (m) shows the number of CD34-, and KDR-positive HPCs; and Panel (r) shows the result of flow cytometry. Panels (n) to (p) and Panel (s) show the results of flow cytometry of hematopoietic progenitor cells obtained by differentiation induction from CB-A11 on Day 6. Exclusion of dead cells and debris was carried out by DAPI staining. Panel (n) shows the total cell number; Panel (o) shows the ratio of CD34-, and KDR-positive HPCs; Panel (p) shows the number of CD34-, and KDR-positive HPCs; and Panel (s) shows the result of flow cytometry. All data are representative data each of which is based on at least three independent experiments. In the flow cytometry analysis, the following antibodies were used: KDR (CD309)-Alexa fluor 647 (Biolegend), CD34-PE (BD Biosciences), CD43-APC (BD Biosciences), and CD45-PE (BD Biosciences).

FIG. 4 is a photograph showing the result of imaging with a scanning electron microscope (SEM). Panel (a) shows an image of a CS before plating of cells. Panel (b) shows an image of a CS on Day 41 after differentiation induction from small clusters of PSCs. Panel (c) shows an image of a CS on Day 21 after differentiation induction from single PSCs. Panels (d) and (e) show images of cells on Day 41 obtained by differentiation induction from small clusters of PSCs. Panels (f) and (g) show images of cells on Day 21 obtained by differentiation induction from single PSCs. As the PSCs, KhES1 was used in all cases.

FIG. 5 shows the results of culture using a plurality of CSs in a flask. Panel (a) shows the number of hematopoietic cells collected after the culture. Panels (b) and (c) show the results on myeloid cells obtained by the culture. Panel (b) shows the result of flow cytometry, and Panel (c) shows a photograph showing the result of Giemsa staining. In the flow cytometry analysis in Panel (b), the following antibodies were used: CD43-APC (BD Biosciences) and CD45-PE (BD Biosciences). The scale bar in Panel (c) represents 20 μm.

FIG. 6 shows the results of PCR for hematopoietic cells induced from small clusters of undifferentiated pluripotent stem cells (PSCs) using a CS. Cells before the differentiation induction (DO) and on Day 6 after the differentiation induction (D6) were subjected to measurement of the gene expression levels of ZFP42 and Nanog as pluripotent cell-specific markers, T and MIXL1 as mesodermal progenitor cell-specific markers, RUNX1 as a hematopoietic progenitor cell-specific marker, and APLNR and CDH5 as endothelial progenitor cell-specific markers.

FIG. 7 shows photographs showing the results of immunostaining of a CS after differentiation induction. Panel (a) shows the sites in the CS where the immunostaining was carried out. Panel (b) shows an immunostaining image obtained using an anti-CD45 antibody at the site B in Panel (a). Panel (c) shows an image obtained by hematoxylin-eosin staining, an image obtained by Masson trichrome staining, an immunostaining image obtained using an anti-CD34 antibody, and an immunostaining image obtained using an anti-CD45 antibody, at the site C in Panel (a).

### EMBODIMENTS FOR CARRYING OUT THE INVENTION

The present invention provides a novel method for producing hematopoietic cells, and a therapeutic agent for blood diseases containing a hematopoietic cell obtained by this method. The present invention also provides a novel method for producing mesodermal cells, and mesodermal cells supported by a three-dimensional support obtained by this method. The present invention also provides a culture vessel retaining a plurality of three-dimensional supports supporting mesodermal cells. In one embodiment of the present invention, the cells produced by the method of the present invention may be cells obtained by differentiation induction from pluripotent stem cells. Examples of the pluripotent stem cells include, but are not limited to, the following cells.

### <Pluripotent Stem Cells>

The pluripotent stem cells (also referred to as “PSCs”) which may be used in the present invention are stem cells having pluripotency that allows the cells to differentiate into any cells existing in the living body, as well as growth ability. Examples of the pluripotent stem cells include, but are not limited to, embryonic stem (ES) cells, embryonic stem cells derived from a cloned embryo obtained by nuclear transfer (“ntES cells”), germline stem cells (“GS cells”), embryonic germ cells (“EG cells”), multilineage-differentiating stress enduring cells (Muse cells), and induced pluripotent stem (iPS) cells. Preferred pluripotent stem cells are ES cells, ntES cells, and iPS cells.

### (A) Embryonic Stem Cells

ES cells are stem cells established from the inner cell mass of an early embryo (for example, blastocyst) of a mammal such as human or mouse, which cells have pluripotency and growth ability by self-renewal.

ES cells are embryo-derived stem cells originated from the inner cell mass of a blastocyst, which is an embryo formed following the 8-cell stage and the morula stage of a fertilized egg, and have an ability to differentiate into any cells constituting an adult, that is, the so-called pluripotency of differentiation, and growth ability by self-renewal. ES cells were discovered in mouse in 1981 (M. J. Evans and M. H. Kaufman (1981), Nature 292: 154-156), and this was followed by establishment of ES cell lines of primates such as human and monkey (J. A. Thomson et al. (1998), Science 282: 1145-1147; J. A. Thomson et al. (1995), Proc. Natl. Acad. Sci. USA, 92: 7844-7848; J. A. Thomson et al. (1996), Biol. Reprod., 55: 254-259; and J. A. Thomson and V. S. Marshall (1998), Curr. Top. Dev. Biol., 38: 133-165).

ES cells can be established by removing the inner cell mass from the blastocyst of a fertilized egg of a subject animal, followed by culturing the inner cell mass on fibroblasts as feeders. The cells can be maintained by subculture using a medium supplemented with a substance(s) such as leukemia inhibitory factor (LIF) and/or basic fibroblast growth factor (bFGF). Methods of establishment and maintenance of human and monkey ES cells are described in, for example, U.S. Pat. No. 5,843,780 B; Thomson J A, et al. (1995), Proc Natl. Acad. Sci. U S A. 92: 7844-7848; Thomson J A, et al. (1998), Science. 282: 1145-1147; H. Suemori et al. (2006), Biochem. Biophys. Res. Commun., 345: 926-932; M. Ueno et al. (2006), Proc. Natl. Acad. Sci. USA, 103: 9554-9559; H. Suemori et al. (2001), Dev. Dyn., 222: 273-279; H. Kawasaki et al. (2002), Proc. Natl. Acad. Sci. USA, 99: 1580-1585; and Klimanskaya I, et al. (2006), Nature. 444: 481-485.

In terms of the medium for preparation of ES cells, human ES cells can be maintained, for example, using DMEM/F-12 medium supplemented with 0.1 mM 2-mercaptoethanol, 0.1 mM non-essential amino acids, 2 mM L-glutamic acid, 20% KSR, and 4 ng/ml bFGF at 37° C. under a moist atmosphere of 5% CO₂ (H. Suemori et al. (2006), Biochem. Biophys. Res. Commun., 345: 926-932). ES cells need to be subcultured every 3 to 4 days, and the subculture can be carried out using, for example, 0.25% trypsin and 0.1 mg/ml collagenase IV in PBS supplemented with 1 mM CaCl₂ and 20% KSR.

Selection of ES cells can be generally carried out by the Real-Time PCR method using as an index/indices expression of a gene marker(s) such as alkaline phosphatase, Oct-3/4, and/or Nanog. In particular, for selection of human ES cells, expression of a gene marker(s) such as OCT-3/4, NANOG, and/or ECAD can be used as an index (E. Kroon et al. (2008), Nat. Biotechnol., 26: 443-452).

In terms of human ES cell lines, for example, WA01(H1) and WA09(H9) can be obtained from WiCell Research Institute, and KhES-1, KhES-2, and KhES-3 can be obtained from Institute for Frontier Medical Sciences, Kyoto University (Kyoto, Japan).

### (B) Germline Stem Cells

Germline stem cells are pluripotent stem cells derived from testis, and play a role as the origin for spermatogenesis. Similarly to ES cells, these cells can be induced to differentiate into various series of cells, and, for example, have a property to enable preparation of a chimeric mouse by transplanting the cells to a mouse blastocyst (M. Kanatsu-Shinohara et al. (2003) Biol. Reprod., 69: 612-616; K. Shinohara et al. (2004), Cell, 119: 1001-1012). Germline stem cells are capable of self-renewal in a medium containing glial cell line-derived neurotrophic factor (GDNF), and, by repeating subculture under the same culture conditions as those for ES cells, germline stem cells can be obtained (Masanori Takehashi et al. (2008), Experimental Medicine, 26(5) (extra edition), 41-46, Yodosha (Tokyo, Japan)).

### (C) Embryonic Germ Cells

Embryonic germ cells are established from fetal primordial germ cells, and have pluripotency similarly to ES cells. They can be established by culturing primordial germ cells in the presence of substances such as LIF, bFGF, and stem cell factor (Y. Matsui et al. (1992), Cell, 70: 841-847; J. L. Resnick et al. (1992), Nature, 359: 550-551).

### (D) Induced Pluripotent Stem Cells

Induced pluripotent stem (iPS) cells can be prepared by introducing specific reprogramming factors into somatic cells, which reprogramming factors are in the form of DNA or protein. iPS cells are somatic cell-derived artificial stem cells having properties almost equivalent to those of ES cells, such as pluripotency of differentiation and growth ability by self-renewal (K. Takahashi and S. Yamanaka (2006) Cell, 126: 663-676; K. Takahashi et al. (2007), Cell, 131: 861-872; J. Yu et al. (2007), Science, 318: 1917-1920; Nakagawa, M. et al., Nat. Biotechnol. 26: 101-106 (2008); WO 2007/069666). The reprogramming factors may be constituted by genes or gene products thereof, or non-coding RNAs, which are expressed specifically in ES cells; or genes or gene products thereof, non-coding RNAs, or low molecular weight compounds, which play important roles in maintenance of the undifferentiated state of ES cells. Examples of the genes included in the reprogramming factors include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, Sall1, Sall4, Esrrb, Nr5a2, Tbx3, and Glis1. These reprogramming factors may be used individually, or two or more of these may be used in combination. Examples of the combination of the reprogramming factors include those described in WO 2007/069666; WO 2008/118820; WO 2009/007852; WO 2009/032194; WO 2009/058413; WO 2009/057831; WO 2009/075119; WO 2009/079007; WO 2009/091659; WO 2009/101084; WO 2009/101407; WO 2009/102983; WO 2009/114949; WO 2009/117439; WO 2009/126250; WO 2009/126251; WO 2009/126655; WO 2009/157593; WO 2010/009015; WO 2010/033906; WO 2010/033920; WO 2010/042800; WO 2010/050626; WO 2010/056831; WO 2010/068955; WO 2010/098419; WO 2010/102267; WO 2010/111409; WO 2010/111422; WO 2010/115050; WO 2010/124290; WO 2010/147395; WO 2010/147612; Huangfu D, et al. (2008), Nat. Biotechnol., 26: 795-797; Shi Y, et al. (2008), Cell Stem Cell, 2: 525-528; Eminli S, et al. (2008), Stem Cells. 26: 2467-2474; Huangfu D, et al. (2008), Nat Biotechnol. 26: 1269-1275; Shi Y, et al. (2008), Cell Stem Cell, 3, 568-574; Zhao Y, et al. (2008), Cell Stem Cell, 3: 475-479; Marson A, (2008), Cell Stem Cell, 3, 132-135; Feng B, et al. (2009), Nat Cell Biol. 11: 197-203; R. L. Judson et al., (2009), Nat. Biotech., 27: 459-461; Lyssiotis C A, et al. (2009), Proc Natl Acad Sci U S A. 106: 8912-8917; Kim J B, et al. (2009), Nature. 461: 649-643; Ichida J K, et al. (2009), Cell Stem Cell. 5: 491-503; Heng J C, et al. (2010), Cell Stem Cell. 6: 167-74; Han J, et al. (2010), Nature. 463: 1096-100; Mali P, et al. (2010), Stem Cells. 28: 713-720; and Maekawa M, et al. (2011), Nature. 474: 225-9.

Examples of the above-described reprogramming factors also include histone deacetylase (HDAC) inhibitors [for example, low molecular weight inhibitors such as valproic acid (VPA), trichostatin A, sodium butyrate, MC 1293, and M344; and nucleic acid-type expression inhibitors such as siRNAs and shRNAs against HDAC (e.g., HDAC1 siRNA Smartpool (registered trademark) (Millipore) and HuSH 29mer shRNA Constructs against HDAC1 (OriGene))], MEK inhibitors (for example, PD184352, PD98059, U0126, SL327, and PD0325901), Glycogen synthase kinase-3 inhibitors (for example, Bio and CHIR99021), DNA methyltransferase inhibitors (for example, 5′-azacytidine), histone methyltransferase inhibitors (for example, low molecular weight inhibitors such as BIX-01294; and nucleic acid-type expression inhibitors such as siRNAs and shRNAs against Suv39h1, Suv39h2, SetDB1, and G9a), L-channel calcium agonists (for example, Bayk8644), butyric acid, TGFβ inhibitors or ALK5 inhibitors (for example, LY364947, SB431542, 616453, and A-83-01), p53 inhibitors (for example, siRNAs and shRNAs against p53), ARID3A inhibitors (for example, siRNAs and shRNAs against ARID3A), miRNAs such as miR-291-3p, miR-294, miR-295, and mir-302, Wnt Signaling (for example, soluble Wnt3a), neuropeptide Y, prostaglandins (for example, prostaglandin E2 and prostaglandin J2), hTERT, SV40LT, UTF1, IRX6, GLIS1, PITX2, and DMRTB1, which are employed for enhancing the establishment efficiency, and, in the present description, these factors employed for the purpose of enhancement of the establishment efficiency are not particularly distinguished from reprogramming factors.

In cases where the reprogramming factors are in the form of protein, the reprogramming factors may be introduced into somatic cells by a method such as lipofection, fusion with a cell membrane-permeable peptide (e.g., HIV-derived TAT or polyarginine), or microinjection.

In cases where the reprogramming factors are in the form of DNA, the reprogramming factors may be introduced into somatic cells by a method such as use of a vector including virus, plasmid, and artificial chromosome vectors; lipofection; use of liposome; or microinjection. Examples of the virus vectors include retrovirus vectors, lentivirus vectors (these are described in Cell, 126, pp. 663-676, 2006; Cell, 131, pp. 861-872, 2007; and Science, 318, pp. 1917-1920, 2007), adenovirus vectors (Science, 322, 945-949, 2008), adeno-associated virus vectors, and Sendai virus vectors (WO 2010/008054). Examples of the artificial chromosome vectors include human artificial chromosomes (HACs), yeast artificial chromosomes (YACs), and bacterial artificial chromosomes (BACs, PACs). Examples of the plasmid which may be used include plasmids for mammalian cells (Science, 322: 949-953, 2008). The vectors may contain a regulatory sequence(s) such as a promoter, enhancer, ribosome binding sequence, terminator, and/or polyadenylation site to enable expression of the nuclear reprogramming factors; and, as required, a sequence of a selection marker such as a drug resistance gene (e.g., kanamycin-resistant gene, ampicillin-resistant gene, or puromycin-resistant gene), thymidine kinase gene, or diphtheria toxin gene; a gene sequence of a reporter such as the green-fluorescent protein (GFP), β-glucuronidase (GUS), or FLAG; and/or the like. Further, in order to remove, after introduction of the above vector into somatic cells, the genes encoding the reprogramming factors, or both the promoters and the genes encoding the reprogramming factors linked thereto, the vector may have LoxP sequences upstream and downstream of these sequences.

In cases where the reprogramming factors are in the form of RNA, each reprogramming factor may be introduced into somatic cells by a method such as lipofection or microinjection, and an RNA in which 5-methylcytidine and pseudouridine (TriLink Biotechnologies) are incorporated may be used in order to suppress degradation (Warren L, (2010) Cell Stem Cell. 7:618-630).

Examples of the medium for induction of the iPS cells include DMEM, DMEM/F12, and DME media supplemented with 10 to 15% FBS (these media may further contain LIF, penicillin/streptomycin, puromycin, L-glutamine, non-essential amino acids, β-mercaptoethanol, and/or the like, if appropriate). Other examples of the medium for induction of the iPS cells include commercially available media [for example, a medium for culturing mouse ES cells (TX-WES medium, Thromb-X), medium for culturing primate ES cells (medium for primate ES/iPS cells, ReproCELL), and serum-free medium (mTeSR, Stemcell Technology)].

Examples of the culture method include a method wherein somatic cells and reprogramming factors are brought into contact with each other at 37° C. in the presence of 5% CO₂ on DMEM or DMEM/F12 medium supplemented with 10% FBS, and the cells are cultured for about 4 to 7 days, followed by plating the cells on feeder cells (e.g., mitomycin C-treated STO cells or SNL cells) and starting culture in a bFGF-containing medium for culturing primate ES cells about 10 days after the contact between the somatic cells and the reprogramming factors, thereby allowing iPS-like colonies to appear about 30 to about 45 days after the contact, or later.

Alternatively, the cells may be cultured at 37° C. in the presence of 5% CO₂ on feeder cells (e.g., mitomycin C-treated STO cells or SNL cells) in DMEM medium supplemented with 10% FBS (this medium may further contain LIF, penicillin/streptomycin, puromycin, L-glutamine, non-essential amino acids, β-mercaptoethanol, and/or the like, if appropriate) for about 25 to about 30 days or longer, to allow ES-like colonies to appear. Preferred examples of the culture method include a method wherein the somatic cells themselves to be reprogrammed are used instead of the feeder cells (Takahashi K, et al. (2009), PLoS One. 4: e8067; or WO 2010/137746), and a method wherein an extracellular matrix (e.g., Laminin-5 (WO 2009/123349) or Matrigel (BD)) is used instead.

Other examples of the culture method include a method wherein culture is carried out using a serum-free medium (Sun N, et al. (2009), Proc Natl Acad Sci U S A. 106:15720-15725). In order to enhance the establishment efficiency, the iPS cells may be established under low oxygen conditions (at an oxygen concentration of 0.1% to 15%) (Yoshida Y, et al. (2009), Cell Stem Cell. 5: 237-241 or WO 2010/013845).

During the culture, the medium is replaced with fresh medium once every day from Day 2 of the culture. The number of the somatic cells used for nuclear reprogramming is not restricted, and usually within the range of about 5×10³ to about 5×10⁶ cells per 100-cm² area on the culture dish.

iPS cells may be selected based on the shape of each formed colony. In cases where a drug resistance gene to be expressed in conjunction with a gene expressed in reprogrammed somatic cells (e.g., Oct3/4 or Nanog) is introduced as a marker gene, established iPS cells can be selected by culturing the cells in a medium containing the corresponding drug (selection medium). Further, iPS cells can be selected by observation under a fluorescence microscope in cases where the marker gene is the gene of a fluorescent protein; by adding a luminescent substrate in cases where the marker gene is the gene of luciferase; or by adding a coloring substrate in cases where the marker gene is the gene of a coloring enzyme.

The term “somatic cells” used in the present description means any animal cells (preferably cells of mammals including human) excluding germ-line cells and totipotent cells such as eggs, oocytes, and ES cells. Examples of the somatic cells include, but are not limited to, any of fetal somatic cells, neonatal somatic cells, and healthy or diseased mature somatic cells, as well as any of primary cultured cells, subcultured cells, and established cell lines. Specific examples of the somatic cells include (1) tissue stem cells (somatic stem cells) such as neural stem cells, hematopoietic stem cells, mesenchymal stem cells, and dental pulp stem cells; (2) tissue progenitor cells; and (3) differentiated cells such as lymphocytes, epithelial cells, endothelial cells, muscle cells, fibroblasts (skin cells and the like), hair cells, hepatic cells, gastric mucosal cells, enterocytes, spleen cells, pancreatic cells (pancreatic exocrine cells and the like), brain cells, lung cells, kidney cells, and adipocytes.

In cases where iPS cells are used as a material for the cells to be transplanted, somatic cells whose HLA genotype is the same or substantially the same as that of the individual to which the cells are to be transplanted are preferably used in view of prevention of the rejection reaction. The term “substantially the same” herein means that the HLA genotype is matching to an extent at which the immune reaction against the transplanted cells can be suppressed with an immunosuppressive agent. For example, the somatic cells have matched HLA types at the 3 loci HLA-A, HLA-B, and HLA-DR, or at the 4 loci further including HLA-C.

(E) ES Cells Derived from Cloned Embryo Obtained by Nuclear Transfer

ntES cells are ES cells derived from a cloned embryo prepared by the nuclear transfer technique, and have almost the same properties as those of ES cells derived from fertilized eggs (T. Wakayama et al. (2001), Science, 292:740-743; S. Wakayama et al. (2005), Biol. Reprod., 72:932-936; J. Byrne et al. (2007), Nature, 450:497-502). That is, an ntES (nuclear transfer ES) cell is an ES cell established from the inner cell mass of a blastocyst derived from a cloned embryo obtained by replacement of the nucleus of an unfertilized egg with the nucleus of a somatic cell. For preparation of an ntES cell, the combination of the nuclear transfer technique (J. B. Cibelli et al. (1998), Nature Biotechnol., 16: 642-646) and the ES cell preparation technique is employed (Sayaka Wakayama et al. (2008), Experimental Medicine 26(5) (extra edition), pp. 47-52). In nuclear transfer, reprogramming can be achieved by injecting the nucleus of a somatic cell into a mammalian enucleated unfertilized egg and culturing the resultant for several hours.

### (F) Multilineage-Differentiating Stress Enduring Cells (Muse Cells)

Muse cells are pluripotent stem cells produced by the method described in WO 2011/007900. More specifically, Muse cells are cells having pluripotency obtained by subjecting fibroblasts or bone marrow stromal cells to trypsin treatment for a long period, preferably to trypsin treatment for 8 hours or 16 hours, followed by suspension culture of the treated cells. Muse cells are positive for SSEA-3 and CD105.

<Method for Inducing Mesodermal Cells from Pluripotent Stem Cells>

In the present description, the term “mesodermal cells” means cells constituting the mesoderm, which cells are capable of producing, during the process of development, the body cavity and mesothelium lining it, muscles, skeletons, dermis, connective tissues, heart/blood vessels (including vascular endothelium), blood (including blood cells), lymph vessels and spleen, kidney and ureter, and gonads (testis, uterus, and gonadal epithelium). The “mesodermal cells” in the present invention can be identified based on expression of one or more of markers such as T (which is the same as Brachyury), VEGF receptor-2 (KDR), FOXF1, FLK1, BMP4, MOX1, SDF1, and CD34. The mesodermal cells preferably express KDR and CD34. Unless otherwise specified, the “mesodermal cells” in the present invention may include hematopoietic stem cells and hematopoietic progenitor cells, which have a capacity to differentiate into hematopoietic cells.

In the present invention, the term “hematopoietic stem cells” means cells which are capable of producing mature blood cells such as T cells, B cells, erythrocytes, platelets, eosinophils, monocytes, neutrophils, or basophils, and have an ability of self-renewal. In the present invention, the term “hematopoietic progenitor cells” (also referred to as “HPCs”) means cells whose differentiation has progressed compared to “hematopoietic stem cells”, and whose direction of differentiation has been determined. These cells can be detected based on expression of one or more of markers such as KDR, CD34, CD90, and CD117, although the markers are not limited to these. In the present description, “hematopoietic progenitor cells” are not distinguished from “hematopoietic stem cells” unless otherwise specified.

The mesodermal cells obtained by the differentiation induction in the present invention may be provided as a cell population containing another type of cells, or may be a purified population. In cases where the mesodermal cells are a cell population containing another type of cells, mesodermal cells are contained in the cell population at a ratio of preferably not less than 30%, more preferably not less than 50%.

In the present invention, the differentiation induction into mesodermal cells is carried out by culturing pluripotent stem cells in contact with a three-dimensional support. The “three-dimensional support” in the present invention may be any three-dimensional substance that can retain cells in a liquid culture (that is, a substance that provides a scaffold for cells). Examples of the “three-dimensional support” include, but are not limited to, biomaterials such as collagen sponges, agarose gel, gelatin, chitosan, hyaluronic acid, proteoglycan, PGA, PLA, and PLGA; and mixtures thereof. As the “three-dimensional support”, a collagen sponge is preferably used.

The collagen sponge preferably has a reinforcing material for the purpose of increasing its strength. Examples of the reinforcing material for the collagen sponge include, but are not limited to, glycolic acid, lactic acid, dioxanone, and caprolactone, and their copolymers; and knitted fabrics, woven fabrics, non-woven fabrics, and other sheet-shaped fiber materials thereof. A preferred reinforcing material in the present invention is a polyethylene terephthalate fiber. The method for the reinforcement with the reinforcing material is not limited. For example, one or both surfaces of a collagen sponge may be laminated with a reinforcing material, or formation of a reinforcing material may be allowed to occur in a collagen sponge during production of the collagen sponge. The collagen sponge in the present invention can be obtained from, for example, MedGEL (#PETcol-24W).

The three-dimensional support in the present invention preferably has a porous structure so that cells can enter into the inside thereof. In such cases, the cells may be present on a surface(s), and/or in the inside, of the three-dimensional support, and may be subjected to a differentiation induction operation at the position(s). In cases where the three-dimensional support is a collagen sponge, the pore size of the collagen sponge is within the range of, for example, 5 μm to 1000 μm, preferably 50 μm to 500 μm. The pore size may be more preferably 200 μm.

In the present description, the term “contact between (pluripotent stem/mesodermal) cells and a three-dimensional support” means that the corresponding cells and the three-dimensional support are positioned close to each other so that a certain interaction can occur between them.

Accordingly, in the present description, the term “culturing (pluripotent stem/mesodermal) cells in contact with a three-dimensional support” means that the corresponding cells are cultured in a state where the cells are positioned close to the three-dimensional support so that the cells and the support can have a certain interaction with each other. Examples of the “contact between (pluripotent stem/mesodermal) cells and a three-dimensional support” include, but are not limited to, physical contacts and chemical contacts. Preferred examples of the contact include binding to the support via receptors present in the cell surface.

The method for inducing differentiation of the pluripotent stem cells into the mesodermal cells is not restricted, and, for example, the following method may be used.

In the present invention, the medium to be used for the induction of the mesodermal cells may be prepared using, as a basal medium, a medium for use in culture of animal cells. Examples of the basal medium include IMDM, Medium 199, mTeSR1 medium, Eagle's Minimum Essential Medium (EMEM), αMEM, Dulbecco's modified Eagle's Medium (DMEM), Ham's F12 medium, RPMI 1640 medium, Fischer's medium, StemPro34 (Invitrogen), Mouse Embryonic fibroblast conditioned medium (MEF-CM), and mixed media thereof. In the present invention, the basal medium is preferably mTeSR1 medium and/or StemPro34 medium. The medium may contain serum, or may be serum-free. The medium may contain, for example, if necessary, one or more of serum replacements such as albumin, transferrin, Knockout Serum Replacement (KSR) (serum replacement for FBS in ES cell culture), N2 supplement (Invitrogen), B27 supplement (Invitrogen), fatty acids, insulin, collagen precursor, trace elements, 2-mercaptoethanol (2ME), and thiolglycerol, and may also contain one or more of substances such as lipids, amino acids, L-glutamine, Glutamax (Invitrogen), non-essential amino acids, vitamins, growth factors, low-molecular-weight compounds, antibiotics, antioxidants, pyruvic acid, buffers, and inorganic salts.

In the present process, the medium may further contain a ROCK inhibitor. The ROCK inhibitor is not limited as long as it can suppress the function of Rho kinase (ROCK). Examples of ROCK inhibitors that may be used in the present invention include Y-27632.

The culture temperature is not limited, and may be about 30 to 40° C., preferably about 37° C. The culture is carried out in an atmosphere of CO₂-containing air. The CO₂ concentration is about 2 to 5%, preferably 5%.

Examples of factors for inducing the differentiation into the mesodermal cells include, but are not limited to, BMP4, VEGF, bFGF, and SCF. Examples of such factors also include any factors which are known to be used for inducing differentiation to mesodermal cells, and any factors which are identified to induce differentiation to mesodermal cells in the future.

In the present invention, the differentiation induction into the mesodermal cells may be carried out by, for example, the steps of:

(i) culturing pluripotent stem cells in contact with a three-dimensional support in a medium containing BMP4; and

(ii) culturing cells obtained in Step (i) in a medium containing VEGF, bFGF, and SCF.

A basal medium to be used in the Step (i) may be preferably mTeSR1 medium, and a basal medium to be used in the Step (ii) may be preferably StemPro34 medium.

The concentrations of the factors for inducing the differentiation into the mesodermal cells in the medium are not limited as long as the induction of the cells of interest is possible.

The concentration of BMP4 in the medium in the Step (i) is, for example, within the range of 1 ng/ml to 200 ng/ml, such as 1 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, or 200 ng/ml, but the concentration is not limited to these. The concentration of BMP4 is preferably 80 ng/ml.

The concentration of VEGF in the medium in the Step (ii) is, for example, within the range of 1 ng/ml to 200 ng/ml, such as 1 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, or 200 ng/ml, but the concentration is not limited to these. The concentration of VEGF is preferably 80 ng/ml.

The concentration of bFGF in the medium in the Step (ii) is, for example, within the range of 1 ng/ml to 100 ng/ml, such as 1 ng/ml, 5 ng/ml, 10 ng/ml, 15 ng/ml, 20 ng/ml, 25 ng/ml, 30 ng/ml, 35 ng/ml, 40 ng/ml, 45 ng/ml, 50 ng/ml, 55 ng/ml, 60 ng/ml, 65 ng/ml, 70 ng/ml, 75 ng/ml, 80 ng/ml, 85 ng/ml, 90 ng/ml, 95 ng/ml, or 100 ng/ml, but the concentration is not limited to these. The concentration of bFGF is preferably 25 ng/ml.

The concentration of SCF in the medium in the Step (ii) is, for example, within the range of 1 ng/ml to 250 ng/ml, such as 1 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, 200 ng/ml, 210 ng/ml, 220 ng/ml, 230 ng/ml, 240 ng/ml, or 250 ng/ml, but the concentration is not limited to these. The concentration of SCF is preferably 100 ng/ml.

The culture period in the Step (i) is, for example, not more than 10 days, preferably 1 to 5 days, especially preferably 3 days.

The culture period in the Step (ii) is, for example, not more than 5 days, preferably 0.5 to 3 days, especially preferably 1 day.

Pluripotent stem cells to be cultured in the Step (i) may be in the form of small clusters formed by aggregation of a plurality of pluripotent stem cells, or may be in the form of singly dissociated cells. The pluripotent stem cells are preferably in the form of singly dissociated cells. Thus, in the present invention, a step of dissociating the pluripotent stem cells into small clusters or single cells may be further included before the Step (i).

The step of dissociating the pluripotent stem cells into small clusters or single cells can be carried out by, for example, a method in which the cells are mechanically dissociated, or a method using a dissociation solution having protease activity and collagenase activity (e.g., Accutase (TM), Accumax (TM), or CTK solution), a dissociation solution having only collagenase activity, or an enzyme-free dissociation solution (e.g., EDTA solution). In cases where the pluripotent stem cells are dissociated into small clusters, the step of dissociation may be preferably the combination of use of CTK solution and mechanical dissociation (for example, dissociation using CTK solution is first carried out, and the cells are then dissociated into small clusters having a desired size by a pipetting operation). In cases where the pluripotent stem cells are dissociated into single cells, the step of dissociation may be preferably the combination of use of CTK solution, use of Accumax, and mechanical dissociation (for example, dissociation using CTK solution is first carried out, and dissociation using Accumax is then carried out, followed by dissociating the cells into single cells by a pipetting operation). In the dissociation step, the dissociation solution may contain a ROCK inhibitor for the purpose of preventing cell death. The ROCK inhibitor is not limited as long as it can suppress the function of Rho kinase (ROCK). Examples of ROCK inhibitors that may be used in the present invention include Y-27632.

In cases where small clusters of pluripotent stem cells are used, the size of each cluster is not limited. The size may be, for example, 100 μm to 1000 μm, preferably 200 μm to 800 μm, especially preferably 300 μm to 500 μm.

In the present invention, pluripotent stem cells (small clusters or single cells) after the dissociation may be first cultured in contact with a three-dimensional support under non-differentiation-inducing conditions for a predetermined period, and may then be cultured under conditions for inducing differentiation into mesodermal cells. Such a preculture step is carried out in order to achieve an appropriate state of adhesion between the pluripotent stem cells and the three-dimensional support. Arbitrary culture conditions may be employed as long as this purpose can be achieved.

Examples of the medium in the preculture step include, but are not limited to, IMDM, Medium 199, mTeSR1 medium, Eagle's Minimum Essential Medium (EMEM), aMEM, Dulbecco's modified Eagle's Medium (DMEM), Ham's F12 medium, RPMI 1640 medium, Fischer's medium, StemPro34 (Invitrogen), Mouse Embryonic fibroblast conditioned medium (MEF-CM), and mixed media thereof. In this step, mTeSR1 medium is preferably used. The medium in this step may further contain a ROCK inhibitor. For example, Y-27632 may be used in this step. The culture temperature is not limited, and may be about 30 to 40° C., preferably about 37° C. The culture is carried out in an atmosphere of CO₂-containing air. The CO₂ concentration is about 2 to 5%, preferably 5%.

In cases where the pluripotent stem cells are small clusters, the culture period in the preculture step is, for example, not more than 5 days, preferably 0.5 to 3 days, especially preferably 1 day. In cases where the pluripotent stem cells are single cells, the culture period in the preculture step is, for example, not more than 6 days, preferably 1 to 4 days, especially preferably 2 days. In the latter cases, the medium is preferably replaced with fresh medium during the preculture step. Examples of the fresh medium for the medium replacement include, but are not limited to, mTeSR1.

In the present invention, the replacement of the medium between the steps may be carried out by replacing only the medium without transferring the three-dimensional support, or may be carried out by transferring the three-dimensional support to a vessel containing fresh medium. The medium replacement is preferably carried out by transferring the three-dimensional support to a vessel containing fresh medium.

<Method for Inducing Hematopoietic Cells from Mesodermal Cells>

In the present invention, the term “hematopoietic cells” means any cells committed to blood lineages. The “hematopoietic cells” in the present invention are preferably arbitrary cells committed to differentiation from mesodermal cells into blood lineages. Examples of the “hematopoietic cells” in the present invention include, but are not limited to, myeloid cells, neutrophils, eosinophils, basophils, erythroid cells, erythroblasts, erythrocytes, monocytic cells, monocytes, macrophages, megakaryocytes, platelets, and dendritic cells. The “hematopoietic cells” in the present invention may be preferably myeloid cells, monocytic cells, or erythroid cells. In the present description, “hematopoietic cells” are not distinguished from “blood cells” unless otherwise specified.

In the present invention, the differentiation induction into hematopoietic cells is carried out by culturing mesodermal cells retained by a three-dimensional support obtained by the above method in a culture vessel. The obtained hematopoietic cells can be collected from the culture supernatant.

(1) Method for Inducing Myeloid Cells from Mesodermal Cells

The “myeloid cells” in the present invention means a series of cells committed to the fate of differentiation from mesodermal cells into myeloid cells. Examples of the myeloid cells include, but are not limited to, neutrophils, eosinophils, and basophils. The “neutrophils” in the present invention are a kind of granulocytes having special granules which can be stained with a neutral dye. The “eosinophils” in the present invention are cells having major basic protein (MBP), eosinophil cationic protein (ECP), eosinophil peroxidase (EPO), and eosinophil-derived neurotoxin (EDN) in their granules. The “eosinophils” are preferably cells having capacity to release EDN by stimulation with secretory immunoglobulin A (sIgA), and capacity to migrate by stimulation with IL-5, eotaxin, and fMLP. The “basophils” in the present invention means cells having large granules that can be stained in dark purple with a basic dye. These cells can be detected based on expression of one or more of markers such as CD43, CD45, CD19, CD13, CD33, and MPO (the types of the markers are not limited). The “myeloid cells” in the present invention may be preferably CD43-, and CD45-positive cells.

The myeloid cells obtained by the differentiation induction in the present invention may be provided as a cell population containing another type of cells, or may be a purified population. In cases where the myeloid cells are a cell population containing another type of cells, myeloid cells are contained in the cell population at a ratio of preferably not less than 30%, more preferably not less than 50%.

In this step, the culture may be carried out using a medium for induction of differentiation of mesodermal cells into myeloid cells. As described above, the mesodermal cells in this step are preferably mesodermal cells obtained by differentiation induction of pluripotent stem cells in contact with a three-dimensional support. Alternatively, mesodermal cells prepared by differentiation induction by an arbitrary method may be brought into contact with a three-dimensional support immediately before this step.

In the present invention, the medium to be used for the induction of the myeloid cells may be prepared using, as a basal medium, a medium for use in culture of animal cells. Examples of the basal medium include IMDM, Medium 199, mTeSR1 medium, Eagle's Minimum Essential Medium (EMEM), aMEM, Dulbecco's modified Eagle's Medium (DMEM), Ham's F12 medium, RPMI 1640 medium, Fischer's medium, StemPro34 (Invitrogen), Mouse Embryonic fibroblast conditioned medium (MEF-CM), and mixed media thereof. In the present step, StemPro34 medium is preferably used. The medium may contain serum, or may be serum-free. The medium may contain, for example, if necessary, one or more of serum replacements such as albumin, transferrin, Knockout Serum Replacement (KSR) (serum replacement for FBS in ES cell culture), N2 supplement (Invitrogen), B27 supplement (Invitrogen), fatty acids, insulin, collagen precursor, trace elements, 2-mercaptoethanol (2ME), and thiolglycerol, and may also contain one or more of substances such as lipids, amino acids, L-glutamine, Glutamax (Invitrogen), non-essential amino acids, vitamins, growth factors, low-molecular-weight compounds, antibiotics, antioxidants, pyruvic acid, buffers, and inorganic salts.

The medium in this step may further contain a ROCK inhibitor. The ROCK inhibitor is not limited as long as it can suppress the function of Rho kinase (ROCK). Examples of ROCK inhibitors that may be used in the present invention include Y-27632.

The culture temperature is not limited, and may be about 30 to 40° C., preferably about 37° C. The culture is carried out in an atmosphere of CO₂-containing air. The CO₂ concentration is about 2 to 5%, preferably 5%.

Examples of factors for inducing the differentiation into the myeloid cells include stem cell factor (SCF), interleukins, thrombopoietin (TPO), and Flt3 ligand. The interleukins herein are proteins secreted from leukocytes, and there are not less than 30 kinds of interleukins such as IL-1, IL-2, IL-3, IL-4, IL-5, IL-6, IL-7, IL-8, and IL-9. In the present invention, the differentiation induction from mesodermal cells into myeloid cells may be preferably carried out by culture in a medium containing at least one of SCF, IL-3, Flt3L, and TPO.

In the present invention, the differentiation induction into the myeloid cells may be carried out by, for example, the step of: culturing mesodermal cells retained by a three-dimensional support in a medium containing SCF, IL-3, Flt3L, and thrombopoietin (TPO).

The basal medium to be used in the above step may be preferably StemPro34 medium.

The concentrations of the factors for inducing the differentiation into the myeloid cells in the medium are not limited as long as the induction of the cells of interest is possible.

The concentration of SCF in the medium is, for example, within the range of 1 ng/ml to 200 ng/ml, such as 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, or 200 ng/ml, but the concentration is not limited to these. The concentration of SCF is preferably 50 ng/ml.

The concentration of IL-3 in the medium is, for example, within the range of 1 ng/ml to 200 ng/ml, such as 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, or 200 ng/ml, but the concentration is not limited to these. The concentration of IL-3 is preferably 50 ng/ml.

The concentration of Flt3L in the medium is, for example, within the range of 1 ng/ml to 200 ng/ml, such as 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, or 200 ng/ml, but the concentration is not limited to these. The concentration of Flt3L is preferably 50 ng/ml.

The concentration of TPO in the medium is, for example, within the range of 1 ng/ml to 20 ng/ml, such as 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 12 ng/ml, 14 ng/ml, 16 ng/ml, 18 ng/ml, or 20 ng/ml, but the concentration is not limited to these. The concentration of TPO is preferably 5 ng/ml.

The culture period in this step is, for example, not more than 60 days, preferably 16 to 41 days, especially preferably 31 days.

(2) Method for Inducing Monocytic Cells from Mesodermal Cells

The “monocytic cells” in the present invention means a series of cells committed to the fate of differentiation from mesodermal cells into monocytic cells. Examples of the monocytic cells include, but are not limited to, monocytes and macrophages. In the present invention, both “monocytes” and “macrophages” are leukocytes. These cells show phagocytosis of foreign substances, and have antigen-presenting capacity. These cells can be detected based on expression of one or more of markers such as CD14, CD16, CD45, and CD68 (the types of the markers are not limited). The “monocytic cells” in the present invention may be preferably CD14-, and CD45-positive cells.

The monocytic cells obtained by the differentiation induction in the present invention may be provided as a cell population containing another type of cells, or may be a purified population. In cases where the monocytic cells are a cell population containing another type of cells, monocytic cells are contained in the cell population at a ratio of preferably not less than 30%, more preferably not less than 50%.

In this step, the culture may be carried out using a medium for induction of differentiation of mesodermal cells into monocytic cells. As described above, the mesodermal cells in this step are preferably mesodermal cells obtained by differentiation induction of pluripotent stem cells in contact with a three-dimensional support. Alternatively, mesodermal cells prepared by differentiation induction by an arbitrary method may be brought into contact with a three-dimensional support immediately before this step.

In the present invention, the medium to be used for the induction of the monocytic cells may be prepared using, as a basal medium, a medium for use in culture of animal cells. Examples of the basal medium include IMDM, Medium 199, mTeSR1 medium, Eagle's Minimum Essential Medium (EMEM), αMEM, Dulbecco's modified Eagle's Medium (DMEM), Ham's F12 medium, RPMI 1640 medium, Fischer's medium, StemPro34 (Invitrogen), Mouse Embryonic fibroblast conditioned medium (MEF-CM), and mixed media thereof. In the present step, StemPro34 medium is preferably used. The medium may contain serum, or may be serum-free. The medium may contain, for example, if necessary, one or more of serum replacements such as albumin, transferrin, Knockout Serum Replacement (KSR) (serum replacement for FBS in ES cell culture), N2 supplement (Invitrogen), B27 supplement (Invitrogen), fatty acids, insulin, collagen precursor, trace elements, 2-mercaptoethanol (2ME), and thiolglycerol, and may also contain one or more of substances such as lipids, amino acids, L-glutamine, Glutamax (Invitrogen), non-essential amino acids, vitamins, growth factors, low-molecular-weight compounds, antibiotics, antioxidants, pyruvic acid, buffers, and inorganic salts.

The medium in this step may further contain a ROCK inhibitor. The ROCK inhibitor is not limited as long as it can suppress the function of Rho kinase (ROCK). Examples of ROCK inhibitors that may be used in the present invention include Y-27632.

The culture temperature is not limited, and may be about 30 to 40° C., preferably about 37° C. The culture is carried out in an atmosphere of CO₂-containing air. The CO₂ concentration is about 2 to 5%, preferably 5%.

Examples of factors for inducing the differentiation into the monocytic cells include, but are not limited to, SCF, IL-3, Flt3L, TPO, M-CSF, and GM-CSF. Examples of such factors also include any factors known to induce differentiation into monocytic cells, and any factors which are identified to induce differentiation into monocytic cells in the future.

In the present invention, the differentiation induction into the monocytic cells may be carried out by, for example, the steps of:

(i) culturing mesodermal cells retained by a three-dimensional support in a medium containing SCF, IL-3, Flt3L, and TPO;

(ii) culturing cells obtained in Step (i) in a medium containing SCF, IL-3, Flt3L, TPO, and M-CSF; and

(iii) culturing cells obtained in Step (ii) in a medium containing Flt3L, M-CSF, and GM-CSF.

The basal medium to be used in the Steps (i) to (iii) may be preferably StemPro34 medium.

The concentrations of the factors for inducing the differentiation into the monocytic cells in the medium are not limited as long as the induction of the cells of interest is possible.

The concentration of SCF in the media in the Steps (i) and (ii) is, for example, within the range of 1 ng/ml to 200 ng/ml, such as 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, or 200 ng/ml, but the concentration is not limited to these. The concentration of SCF is preferably 50 ng/ml.

The concentration of IL-3 in the media in the Steps (i) and (ii) is, for example, within the range of 1 ng/ml to 200 ng/ml, such as 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, or 200 ng/ml, but the concentration is not limited to these. The concentration of IL-3 is preferably 50 ng/ml.

The concentration of Flt3L in the media in the Steps (i) to (iii) is, for example, within the range of 1 ng/ml to 200 ng/ml, such as 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, or 200 ng/ml, but the concentration is not limited to these. The concentration of Flt3L is preferably 50 ng/ml.

The concentration of TPO in the media in the Steps (i) and (ii) is, for example, within the range of 1 ng/ml to 20 ng/ml, such as 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 12 ng/ml, 14 ng/ml, 16 ng/ml, 18 ng/ml, or 20 ng/ml, but the concentration is not limited to these. The concentration of TPO is preferably 5 ng/ml.

The concentration of M-CSF in the media in the Steps (ii) and (iii) is, for example, within the range of 1 ng/ml to 200 ng/ml, such as 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, or 200 ng/ml, but the concentration is not limited to these. The concentration of M-CSF is preferably 50 ng/ml.

The concentration of GM-CSF in the medium in the Step (iii) is, for example, within the range of 1 ng/ml to 100 ng/ml, such as 1 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml, but the concentration is not limited to these. The concentration of GM-CSF is preferably 25 ng/ml.

The culture period in the Step (i) is, for example, not more than 10 days, preferably 2 to 5 days, especially preferably 3 days.

The culture period in the Step (ii) is, for example, not more than 10 days, preferably 2 to 5 days, especially preferably 3 days.

The culture period in the Step (iii) is, for example, not more than 40 days, preferably 8 to 33 days, especially preferably 23 days.

In the present invention, the replacement of the medium between the steps may be carried out by replacing only the medium without transferring the three-dimensional support, or may be carried out by transferring the three-dimensional support to a vessel containing fresh medium. The medium replacement is preferably carried out by transferring the three-dimensional support to a vessel.

(3) Method for Inducing Erythroid Cells from Mesodermal Cells

The “erythroid cells” in the present invention means a series of cells committed to the fate of differentiation from mesodermal cells into erythroid cells. Examples of the erythroid cells include, but are not limited to, erythrocytes. The “erythrocytes” in the present invention means cells rich in hemoglobin, and can be detected based on expression of one or more of markers such as α-globin, ε-globin, γ-globin, and β-globin of hemoglobin, and CD235a. Preferred markers for mature erythrocytes may be α-globin and β-globin (the types of the markers are not limited). In cases where erythrocytes are separated by FACS, CD235a may be a preferred marker. The “erythroid cells” in the present invention may be preferably CD71-, and CD235a-positive cells.

The erythroid cells obtained by the differentiation induction in the present invention may be provided as a cell population containing another type of cells, or may be a purified population. In cases where the erythroid cells are a cell population containing another type of cells, erythroid cells are contained in the cell population at a ratio of preferably not less than 30%, more preferably not less than 50%.

In this step, the culture may be carried out using a medium for induction of differentiation of mesodermal cells into erythroid cells. As described above, the mesodermal cells in this step are preferably mesodermal cells obtained by differentiation induction of pluripotent stem cells in contact with a three-dimensional support. Alternatively, mesodermal cells prepared by differentiation induction by an arbitrary method may be brought into contact with a three-dimensional support immediately before this step.

In the present invention, the medium to be used for the induction of the erythroid cells may be prepared using, as a basal medium, a medium for use in culture of animal cells. Examples of the basal medium include IMDM, Medium 199, mTeSR1 medium, Eagle's Minimum Essential Medium (EMEM), aMEM, Dulbecco's modified Eagle's Medium (DMEM), Ham's F12 medium, RPMI 1640 medium, Fischer's medium, StemPro34 (Invitrogen), Mouse Embryonic fibroblast conditioned medium (MEF-CM), and mixed media thereof. In the present step, StemPro34 medium is preferably used. The medium may contain serum, or may be serum-free. The medium may contain, for example, if necessary, one or more of serum replacements such as albumin, transferrin, Knockout Serum Replacement (KSR) (serum replacement for FBS in ES cell culture), N2 supplement (Invitrogen), B27 supplement (Invitrogen), fatty acids, insulin, collagen precursor, trace elements, 2-mercaptoethanol (2ME), and thiolglycerol, and may also contain one or more of substances such as lipids, amino acids, L-glutamine, Glutamax (Invitrogen), non-essential amino acids, vitamins, growth factors, low-molecular-weight compounds, antibiotics, antioxidants, pyruvic acid, buffers, and inorganic salts.

The medium in this step may further contain a ROCK inhibitor. The ROCK inhibitor is not limited as long as it can suppress the function of Rho kinase (ROCK). Examples of ROCK inhibitors that may be used in the present invention include Y-27632.

The culture temperature is not limited, and may be about 30 to 40° C., preferably about 37° C. The culture is carried out in an atmosphere of CO₂-containing air. The CO₂ concentration is about 2 to 5%, preferably 5%.

Examples of factors for inducing the differentiation into the erythroid cells include stem cell factor (SCF), colony-stimulating factor (CSF), granulocyte colony-stimulating factor (G-CSF), erythropoietin (EPO), interleukins, thrombopoietin (TPO), and Flt3 ligand. The interleukins herein are proteins secreted from leukocytes, and there are not less than 30 kinds of interleukins such as IL-1, IL-2, IL-3, IL-4, IL-5, IL-6, IL-7, IL-8, and IL-9. In the present invention, the differentiation induction from mesodermal cells into erythroid cells may be carried out by culture in a medium containing at least one of EPO, IL-3, and SCF.

In the present invention, the differentiation induction into the erythroid cells may be carried out by, for example, the step of: culturing mesodermal cells retained by a three-dimensional support in a medium containing erythropoietin (EPO) and SCF.

The basal medium to be used in the above step may be preferably StemPro34 medium.

The concentrations of the factors for inducing the differentiation into the erythroid cells in the medium are not limited as long as the induction of the cells of interest is possible.

The concentration of EPO in the medium is preferably within the range of 1 U/ml to 20 U/ml, such as 1 U/ml, 2 U/ml, 3 U/ml, 4 U/ml, 5 U/ml, 6 U/ml, 7 U/ml, 8 U/ml, 9 U/ml, 10 U/ml, 12 U/ml, 14 U/ml, 16 U/ml, 18 U/ml, or 20 U/ml, but the concentration of EPO is not limited to these. The concentration of EPO is preferably 5 U/ml.

The concentration of SCF in the medium is, for example, within the range of 1 ng/ml to 200 ng/ml, such as 1 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 140 ng/ml, 160 ng/ml, 180 ng/ml, or 200 ng/ml, but the concentration is not limited to these. The concentration of SCF is preferably 50 ng/ml.

The culture period in this step is, for example, not more than 60 days, preferably 16 to 41 days, especially preferably 31 days.

<Method for Inducing Hematopoietic Cells from Pluripotent Stem Cells>

In the present invention, hematopoietic cells can be induced from pluripotent stem cells by the combination of the <Method for Inducing Mesodermal Cells from Pluripotent Stem Cells> and the <Method for Inducing Hematopoietic Cells from Mesodermal Cells>. In such cases, examples of the differentiation induction conditions include, but are not limited to, those in the following method.

Examples of the method for inducing myeloid cells from pluripotent stem cells include a method comprising the steps of:

(i) culturing pluripotent stem cells in contact with a three-dimensional support in a medium containing BMP4;

(ii) culturing cells obtained in Step (i) in a medium containing VEGF, bFGF, and SCF; and

(iii) culturing cells obtained in Step (ii) in a medium containing SCF, IL-3, Flt3L, and thrombopoietin (TPO).

Examples of the method for inducing monocytic cells from pluripotent stem cells include a method comprising the steps of:

(i) culturing pluripotent stem cells in contact with a three-dimensional support in a medium containing BMP4;

(ii) culturing cells obtained in Step (i) in a medium containing VEGF, bFGF, and SCF;

(iii) culturing cells obtained in Step (ii) in a medium containing SCF, IL-3, Flt3L, and TPO;

(iv) culturing cells obtained in Step (iii) in a medium containing SCF, IL-3, Flt3L, TPO, and M-CSF; and

(v) culturing cells obtained in Step (iv) in a medium containing Flt3L, M-CSF, and GM-CSF.

Examples of the method for inducing erythroid cells from pluripotent stem cells include a method comprising the steps of:

(i) culturing pluripotent stem cells in contact with a three-dimensional support in a medium containing BMP4;

(ii) culturing cells obtained in Step (i) in a medium containing VEGF, bFGF, and SCF; and

(iii) culturing cells obtained in Step (ii) in a medium containing erythropoietin (EPO) and SCF.

The concentrations of the differentiation-inducing factors, the culture period, and other culture conditions may be those described above, or may be those in an arbitrary technique that has been known in the present field before the application of the present invention.

### <Therapeutic Agent for Diseases>

The present invention provides, as a therapeutic agent for diseases, (A) a graft material containing a three-dimensional support containing a mesodermal cell produced by the method of the present invention, or (B) a therapeutic agent for blood diseases containing a hematopoietic cell produced by the method of the present invention. The mesodermal cell and/or the hematopoietic cell in the three-dimensional support obtained by the method of the present invention may be derived from the patient himself to be treated, or may be derived from another/other individual(s). The mesodermal cell and/or the hematopoietic cell is/are preferably derived from the patient himself to be treated. In cases where the mesodermal cell and/or the hematopoietic cell in the three-dimensional support is/are derived from another/other individual(s), somatic cells are preferably collected from the another/other individual(s) having the same type of HLA from the viewpoint of prevention of rejection.

In the embodiment (A), the agent in the present invention may contain the three-dimensional support alone containing a mesodermal cell, or may contain a buffer, antibiotic, another pharmaceutical additive, and/or the like together with the three-dimensional support containing a mesodermal cell. For example, the agent in the present invention may further contain a scaffold material (scaffold) such as fibronectin, laminin, synthetic polymer (e.g., polylactic acid), and/or the like for the purpose of promoting engraftment of the cells contained in the three-dimensional support in the recipient tissue. The agent in the present invention may also contain an arbitrary cell(s) other than mesodermal cells. In the embodiment (B), the agent in the present invention may contain the induced hematopoietic cell(s) alone, or may contain a buffer, antibiotic, another pharmaceutical additive, and/or arbitrary cell(s) other than hematopoietic cells, together with the hematopoietic cell(s).

The agent in the present invention is effective as a therapeutic agent for a wide range of blood diseases. Specific examples of the diseases to be treated with the therapeutic agent in the present invention include, but are not limited to, congenital anemia; aplastic anemia; autoimmune anemia; myelodysplastic syndrome (MDS); agranulocytosis; hypolymphemia; thrombocytopenia; hematopoietic stem cell and/or hematopoietic progenitor cell cytopenia due to cancers and tumors; hematopoietic stem cell and/or hematopoietic progenitor cell cytopenia due to cancer chemotherapy or radiotherapy; acute radiation syndrome; delayed recovery of hematopoietic stem cells and/or hematopoietic progenitor cells after transplantation of bone marrow, cord blood, or peripheral blood; hematopoietic stem cell and/or hematopoietic progenitor cell cytopenia due to blood transfusion; leukemia (including acute myelocytic leukemia (AML), acute lymphocytic leukemia (ALL), chronic myeloid leukemia (CML), and chronic lymphocytic leukemia (CLL)); malignant lymphoma; multiple myeloma; myeloproliferative disorders; and hereditary blood disorders.

In the present invention, the administration route of the agent to the patient is not limited. In the embodiment (A), the agent is preferably administered to the patient by transplantation. In such cases, the transplantation can be carried out by the same method as that of conventional bone marrow transplantation or cord blood transplantation. In the present invention, the administration (transplantation) of the agent may be carried out by a plurality of times of placement at the same site. In cases where the placement is carried out several times, these operations of placement are preferably carried out at sufficient time intervals to allow engraftment of the desired cells in the tissue. In the embodiment (B), examples of the dosage form include intravenous, subcutaneous, intracutaneous, intramuscular, intraperitoneal, intramedullary, and intracerebral administration. The administration route may be preferably intravenous or intramedullary administration. In the embodiment (B), similarly to the embodiment (A), the hematopoietic cells obtained by the present invention can be used for transplantation therapy. In such cases, the transplantation can be carried out by the same method as that of conventional bone marrow transplantation or cord blood transplantation.

The dose of the agent or the amount of the agent to be transplanted to the patient varies depending on, for example, the type of the disease state to be treated; symptoms and severity of the disease; the age, sex, and/or body weight of the patient; and/or the administration method/transplantation method. Physicians can determine an appropriate dose or amount of transplant by taking the above conditions into account.

### <Large-Scale Culture>

In the present invention, in order to obtain a large amount of hematopoietic cells, the cells may be cultured in a culture vessel containing one or more three-dimensional supports that can be placed in the vessel. Each three-dimensional support to be used in this step is in a state where it is retaining pluripotent stem cells and/or mesodermal cells. For example, the three-dimensional support may be a three-dimensional support in contact with pluripotent stem cells before differentiation induction, or may be a three-dimensional support in a state where pluripotent stem cells in contact therewith have been allowed to differentiate into mesodermal cells. In such cases, the pluripotent stem cells to be used as the origin for the differentiation into the mesodermal cells may be in the form of either small clusters or single cells. The pluripotent stem cells may be preferably in the form of single cells. The culture vessel to be used in this step may be an arbitrary vessel which is usually used in this field. Examples of the culture vessel include, but are not limited to, dishes, flasks, and culture tanks. The culture vessel may be preferably a vessel having a large capacity that enables culture of a large amount of cells. The medium to be used for the large-scale culture may be the same as that used for the above-described differentiation induction into mesodermal cells or hematopoietic cells. For allowing the hematopoietic cell obtained by the above method to grow into more mature hematopoietic cells, a medium for an arbitrary differentiation induction method that has been known by the application date of the present invention may also be used. Examples of the culture conditions that may be employed for the large-scale culture include static culture, shake culture, and spinner culture. From the viewpoint of allowing efficient contact of cells with the medium components, shake culture or spinner culture is preferably used.

Collection of the hematopoietic cell produced may be carried out by collecting the medium containing the hematopoietic cells. By repeating the collection of the medium, a large amount of hematopoietic cells can be collected. The collection of the medium may be manually carried out, or may be carried out using, for example, an apparatus having an automated collection device. The collection is preferably carried out using an apparatus having an automated collection device. Another example of the method for collecting the hematopoietic cells is a method by collection of cells adhering to the three-dimensional support. In such a case, a recovered three-dimensional support is subjected to physical or enzymatic treatment to dissociate the cells from the three-dimensional support. By this, the cells can be collected. Either one or both of the collection of the hematopoietic cells from the medium and the collection of the cells from the three-dimensional support may be carried out. Since most floating cells are hematopoietic cells, the hematopoietic cells can be more efficiently collected by the collection from the medium. In view of this, the cells are preferably collected by the collection of the medium in which the culture using the three-dimensional support has been carried out. The collected hematopoietic cells may be used as they are, or may be used after purification, depending on the purpose of use.

The present invention is described below more concretely by way of Examples, but the scope of the present invention is not limited to the Examples.

### EXAMPLES

### Example 1

### Pluripotent Stem Cells

The following cell lines were used as human ES cells (KhES1 cell line) and human iPS cells (201B7 cell line, 409B2 cell line, and CB-A11 cell line).

### (1) KhES1 Cell Line

As human ES cells, the KhES1 cell line, which was established by Stem Cell Research Center, Institute for Frontier Medical Sciences, Kyoto University, was used. The culture was carried out by a conventional method (Suemori H, et al. Biochem Biophys Res Commun. 345: 926-32, 2006). The human ES cells were used with approval of Ministry of Education, Culture, Sports, Science and Technology (MEXT).

### (2) 201B7 Cell Line

The 201B7 cell line was prepared by the method described in Takahashi K, et al. Cell. 131: 861-72, 2007.

### (3) 409B2 Cell Line

According to the method described in Okita K., et al., Stem Cells. 2012 Nov. 29, the iPS cell line was prepared by transfecting human skin cells with episomal vectors (pCXLE-hOCT3/4-shp53-F, pCXLE-hSK, and pCXLE-hUL) by electroporation, and culturing the transfected cells on mouse fetal fibroblast feeders treated with mitomycin. The culture was carried out by a conventional method (Takahashi K, et al. Cell. 131: 861-72, 2007 and Nakagawa M, et al. Nat Biotechnol. 26: 101-6, 2008).

### (4) CB-All Cell Line

According to the method described in Yanagimachi, M. D. et al., PLoS ONE, 8(4): e59243, 2013, an iPS cell line was prepared from cord blood hematopoietic cells. The resulting iPS cell line was subjected to maintenance culture on division-inactivated SNL feeder cells in primate ES Cell Medium (ReproCELL) supplemented with 5 ng/mL bFGF, or to maintenance culture on a tissue culture dish coated with growth factor-reduced Matrigel (Becton-Dickinson) in mTeSR1 serum-free medium (STEMCELL Technologies). The medium was replaced every day.

### Preparation of Collagen Sponge

A collagen sponge mechanically reinforced by incorporation of polyethylene terephthalate (PET) fibers (PETCol-24w, hereinafter referred to as “CS”) was purchased from MedGEL44. For removal of air bubbles, six CSs were placed in a 50-mL conical tube (Becton-Dickinson) containing 10 mL of a maintenance medium for pluripotent stem cells, and the tube was then centrifuged at 10,000 rpm for 5 minutes. Thereafter, the CSs were removed using a spatula, and placed in a 24-well plate for use in the following experiment.

### Example 2

Induction of Hematopoietic Cells from Small Clusters of Pluripotent Stem Cells
(1) Induction of Mesodermal Cells from Small Clusters of Pluripotent Stem Cells

In order to study whether or not induction of mesodermal cells from small clusters of pluripotent stem cells is possible under culture conditions using a three-dimensional support (CS), the following experiment was carried out. Briefly, each of the KhES1 cell line, 201B7 cell line, 409B2 cell line, and CB-A11 cell line was treated with CTK solution at room temperature for 1 minute, and then washed twice with phosphate-buffered saline (PBS). Subsequently, each cell line was detached from the culture plate using a scraper, and 1.5 mL mTeSR1 medium was added thereto, followed by collecting the cell line into a 15-mL conical tube (Becton-Dickinson) and dissociating the cells by pipetting such that small clusters with diameters of 300 to 500 μm were formed. The tube was then left to stand for 1 minute to allow the small clusters of the cell line to precipitate. Thereafter, the supernatant was removed, and the small clusters of the cell line were collected. Subsequently, the small clusters of the cell line were plated on a CS placed in a 24-well plate. The CS was prepared by the method in Example 1. Using a maintenance medium for undifferentiated pluripotent stem cells (mTeSR1), the cells were incubated overnight. The CS was then transferred to a 12-well plate containing a differentiation medium (mTeSR1 supplemented with BMP4).

The types of the medium and the cytokines were determined as described previously (Niwa, A. et al. PloS one 6, e22261, 2011 and Yanagimachi, M. D. et al., PLoS ONE, 8(4): e59243, 2013). Briefly, first, on Day 0 to Day 3, the culture was carried out in mTeSR1 medium supplemented with 80 ng/mL BMP4 (R&D Systems), to induce primitive streak cells. Subsequently, on Day 4, the mTeSR1 medium was replaced with StemPro-34 serum-free medium (containing 2 mM glutaMAX (Invitrogen)) supplemented with a cytokine cocktail composed 80 ng/mL VEGF (R&D Systems), 25 ng/mL bFGF (Wako), and 100 ng/mL SCF (R&D Systems).

On Day 6 after the beginning of the differentiation, the cells were collected. Briefly, for collecting adhering cells on the CS, the CS was placed on the side wall of a conical tube (Becton-Dickinson), and the tube was then centrifuged at 7000 rpm for 10 seconds to remove water from the CS. Subsequently, 1 mL of Accmax (Innovative Cell Technologies, Inc.) was added to the CS. After incubation at 37° C. for 10 minutes, 9 mL of PBS was added to the well, and the resulting mixture was mixed. The CS was compressed using a spatula, and then removed from the tube, followed by centrifuging the tube at 1500 rpm for 5 minutes to obtain a cell pellet. For collecting the floating cells, the CS was transferred into another well, and the remaining medium was collected. The collected medium was centrifuged at 1500 rpm for 5 minutes to obtain a cell pellet.

The collected cells were subjected to flow cytometry analysis to measure the ratio of KDR-, and CD34-positive and CD45-negative cells. Briefly, data from the flow cytometry analysis were collected using a MACS Quant™ Analyzer (Miltenyi Biotec), and analyzed using the FlowJo software package (Treestar).

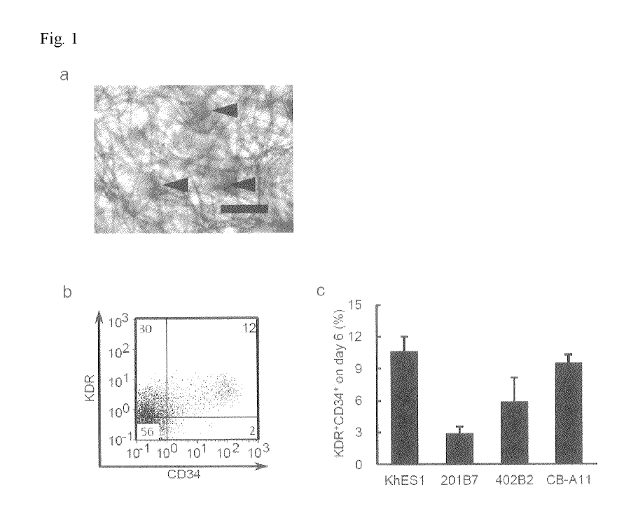

As a result, on Day 6 after the differentiation, KDR-, and CD34-positive HPCs were obtained from all of the KhES1 cell line, 201B7 cell line, 409B2 cell line, and CB-A11 cell line (FIG. 1c). The differentiation induction efficiencies were comparable to those previously reported for cases where the 2D Matrigel method was used (Niwa, A. et al. PloS one 6, e22261, 2011 and Yanagimachi, M. D. et al., PLoS ONE, 8(4): e59243, 2013).

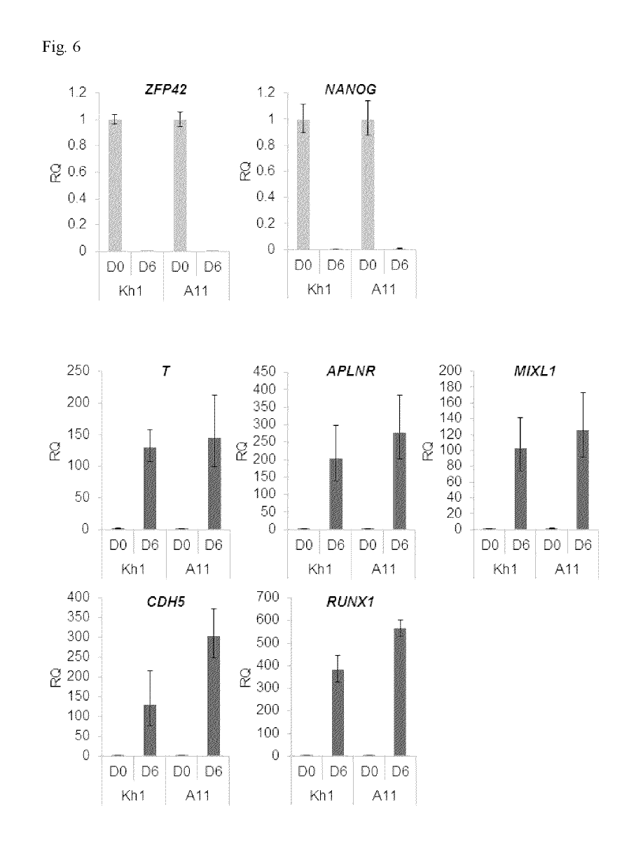

The resulting HPCs (D6) and the pluripotent stem cells before the induction (the KhES1 cell line or the CB-A11 cell line) were subjected to measurement of marker genes (ZFP42 and Nanog as pluripotent stem cell-specific markers, T and MIXL1 as mesodermal progenitor cell-specific markers, RUNX1 as a hematopoietic progenitor cell-specific marker, and APLNR and CDH5 as endothelial progenitor cell-specific markers) by quantitative PCR (FIG. 6). As a result, in the HPCs obtained, expression of ZFP42 or Nanog could be hardly found, and increased expression was found for T, MIXL1, RUNX1, APLNR, and CDH5. From these results, it was confirmed that, as a result of induction into hematopoietic mesodermal progenitor cells, the cells obtained by the present method hardly include residual pluripotent stem cells.

(2) Method for Inducing Hematopoietic Cells from Mesodermal Cells

The mesodermal cells obtained in (1) as described above were cultured in a medium supplemented with a combination of particular cytokines, to allow differentiation induction into particular different hematopoietic cells. Briefly, on Day 6 after the beginning of the differentiation induction, the medium was replaced with StemPro-34 serum-free medium supplemented with different combinations of cytokines, to allow differentiation into particular hematopoietic cell lineages. For induction of myeloid cells, culture was carried out using StemPro-34 serum-free medium supplemented with 50 ng/mL SCF (R&D Systems), 50 ng/mL IL-3 (R&D Systems), 5 ng/mL TPO (R&D Systems), and 50 ng/mL FL3 (R&D Systems). During the culture, medium replacement was carried out every four days. For induction of monocytic cells, culture was carried out using StemPro-34 serum-free medium supplemented with 50 ng/mL SCF (R&D Systems), 50 ng/mL IL-3 (R&D Systems), 5 ng/mL TPO (R&D Systems), and 50 ng/mL FL3 (R&D Systems) from Day 6 to Day 9. On Day 10, the medium was replaced with StemPro-34 serum-free medium supplemented with 50 ng/mL SCF (R&D Systems), 50 ng/mL IL-3 (R&D Systems), 5 ng/mL TPO (R&D Systems), 50 ng/mL M-CSF (R&D Systems), and 50 ng/mL FL3 (R&D Systems). On Day 14, the medium was replaced with StemPro-34 serum-free medium supplemented with 50 ng/mL FL3 (R&D Systems), 25 ng/mL GM-CSF (R&D Systems), and 50 ng/mL M-CSF (R&D Systems). For induction of erythroid cells, culture was carried out using StemPro-34 serum-free medium supplemented with 5 IU/mL EPO (EMD Biosciences), 50 ng/mL IL-3 (R&D Systems), and 50 ng/mL SCF (R&D Systems). During the culture, medium replacement was carried out every other day. From Day 14 after the first beginning of the differentiation induction (Day 0), the CS was transferred to a well containing fresh medium every 2 to 5 days. Every time when the CS was transferred to another well, the medium left after the transfer was repeatedly collected (on Day 22, Day 27, Day 32, Day 37, Day 42, and Day 47).

The collected cells were subjected to flow cytometry analysis. Briefly, data from the flow cytometry analysis were collected using a MACS Quant™ Analyzer (Miltenyi Biotec), and analyzed using the FlowJo software package (Treestar). Giemsa staining was carried out for observation of morphology of the cells obtained. The Giemsa staining was carried out by plating cells on a glass slide using CYTOSPIN 4 (Thermo Scientific), and staining the cells with the May-Grunwald-Giemsa dye (MERCK) according to manufacturer's instructions.

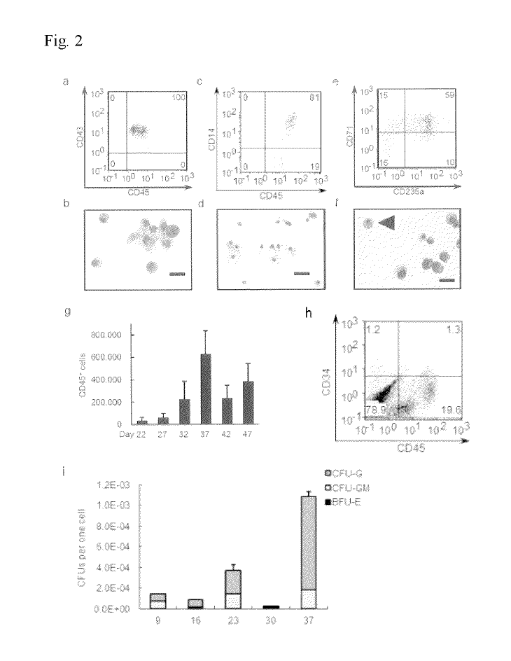

As a result, release of floating hematopoietic cells into the medium began on about Day 20. In the differentiation induction into myeloid cells, almost 100% of the floating cells showed CD43⁺ CD45⁺, which are markers for hematopoietic cells (FIG. 2a), and a part of the cells showed neutrophil-like or macrophage-like morphology (FIG. 2b). Hematopoietic cells could be repeatedly collected during a period of not less than 40 days (FIG. 2c). In the differentiation induction into monocytic cells, the collected live cells were mostly CD14⁺ CD45⁺, and had a monocyte-like/macrophage-like appearance (FIG. 2d and FIG. 2e). In the differentiation induction into erythroid cells, CD71⁺ CD235a⁺ erythroid cells could be obtained (FIG. 2f). These cells had basophilic cytoplasm having a high N/C ratio, similarly to erythroid progenitor cells (FIG. 2g). It was shown, as a whole, that a culture system using a CS enables differentiation of small clusters of pluripotent stem cells into hematopoietic cell lineages.

It was also confirmed, by collecting cells on the CS, that CD34⁺ progenitor cells and CD45⁺ hematopoietic cells were actually cultured (FIG. 2h). As a result of a colony formation test for evaluation of the hematopoietic cells obtained by the present method, it was found that the colonies induced using the CS maintained the colony-forming capacity until Day 37 (FIG. 2i). The obtained cells tended to show induction into myeloid cells.

### Example 3

Induction of Hematopoietic Cells from Single Pluripotent Stem Cells
(1) Induction of Mesodermal Cells from Single Pluripotent Stem Cells

Since the average pore size of the CS is about 200 μm, it was thought that penetration of the small clusters of PSCs into the CS did not occur. In view of this, for utilization of the three-dimensional structure of the CS, a study was carried out to see whether or not differentiation, into hematopoietic cells, of PSCs dissociated into single cells can be supported by the CS. Briefly, each of the KhES1 cell line, 201B7 cell line, 409B2 cell line, and CB-All cell line was treated with CTK solution at room temperature for 1 minute, and then washed twice with phosphate-buffered saline (PBS). Subsequently, using 1 mL of Accumax (Innovative Cell Technologies, Inc.), the cells were treated at 37° C. for 10 minutes, followed by dissociating the cells with the tip of a T1000 pipette, and collecting the cells into a 15-mL conical tube (Becton-Dickinson) containing 9 mL of PBS. Thereafter, the cells were washed twice to remove collagenase contained in Accumax, and centrifugation was then carried out at 1500 rpm for 5 minutes. Subsequently, the cell pellet was suspended in 500 to 1000 μL of mTeSR1 supplemented with 10 mM Y27632 (abcam). At the center of a CS placed in a 24-well plate, 50 μL of the cell suspension containing each cell line was gently added dropwise. The CS was prepared by the method in Example 1. On the next day, 1 mL of a maintenance medium for undifferentiated pluripotent stem cells (mTeSR1) was added to the cells, and culture was further carried out for 1 day, followed by transferring the CS to a 12-well plate containing a differentiation medium (mTeSR1 supplemented with BMP4).

The following differentiation step was carried out by the same method as in Example 2.

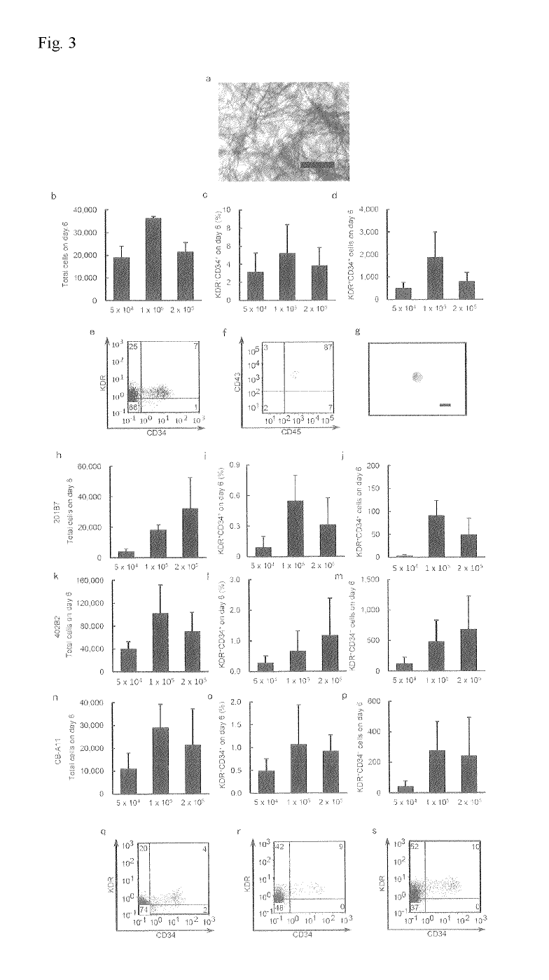

As a result, on Day 6, KDR-, and CD34-positive HPCs were obtained from all of the KhES1 cell line, 201B7 cell line, 409B2 cell line, and CB-A11 cell line (FIG. 3b to FIG. 3e, and FIG. 3g to FIG. 3s).

In order to determine the optimal number of undifferentiated pluripotent stem cells per CS, a study was carried out using several different starting numbers of cells. As a result, the highest production of KDR-, and CD34-positive HPCs could be found for the KhES1 strain when the cell number was 1×10⁵ per CS (FIG. 3b to FIG. 3d). The results were different among the cell lines (FIG. 3b to FIG. 3d, and FIG. 3h to FIG. 3p).

(2) Induction of Hematopoietic Cells from Mesodermal Cells

Differentiation induction into myeloid cells was carried out by the same method as in Example 2. As a result, release of floating hematopoietic cells into the medium began on about Day 20, similarly to the case where small clusters of pluripotent stem cells were used. In the differentiation induction into myeloid cells, the floating live cells showed bone marrow-like surface markers and morphology (FIG. 3f and FIG. 3g).

### Example 4

### Observation by Scanning Microscopy and Immunostaining

In order to evaluate interactions between differentiated cells, and the state of distribution of differentiated cells in the scaffold, observation using a scanning microscope was carried out for hematopoietic cells prepared by differentiation induction from small clusters or single cells by the method of Example 2 or 3, respectively. Briefly, each of the CS before plating of the cells, the CS after differentiation of small clusters of PSCs for 41 days, the CS after differentiation of single PSCs for 21 days, the CS after differentiation of small clusters of PSCs for 41 days, and the CS after differentiation of single PSCs for 21 days was fixed using 4% paraformaldehyde and 2% glutaraldehyde at 4° C. overnight. After fixation with 1% OsO₄ for 3 hours (post-fixation), each CS was dried by dehydration, and coated with a thin film of platinum palladium. Thereafter, each sample was observed using a Hitachi S-4700 scanning electron microscope (Hitachi, Tokyo, Japan). As the PSCs, KhES1 was used in all cases.

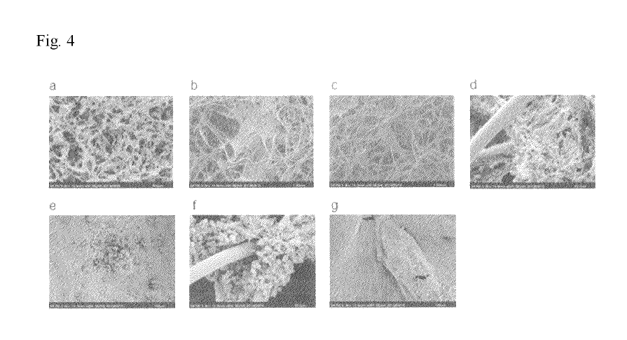

As a result, gradual destruction of collagen fibers during the differentiation was found due to the influence of intracellular collagenase (FIG. 4a to FIG. 4c). When the small clusters of PSCs were allowed to differentiate, aggregation of spherical hematopoietic-like cells occurred such that they surrounded PET fibers (FIG. 4d). Interestingly, these hematopoietic cells were adhering to sheet-like cells cross-linking PET fibers (FIG. 4e). These structures were found also in the cases of differentiation from single cells (FIG. 4f and FIG. 4g). As a whole, the observation using the scanning microscope revealed that the hematopoietic cell niche on the CS is placed based on close positional relationships among PET fibers, hematopoietic cells, and nonhematopoietic supporting cells. Interestingly, the factors constituting this niche were the same as those of an in vitro hematopoietic microenvironment which was reported previously (Leisten I, et al., Biomaterials. 33(6): 1736-47 (2012)).

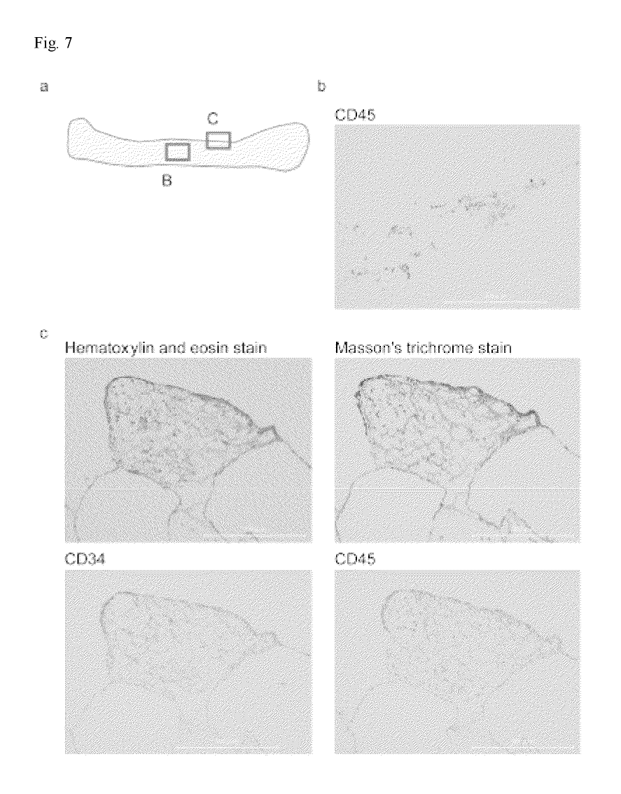

In order to further investigate properties of cells inside the CS, immunostaining was carried out for each CS. As a result, it was found that CD34⁺ cells and collagen fibers formed sac-shaped structures, and that CD45⁺ cells were incorporated in cavities in the CS (FIG. 7a to FIG. 7c).

### Example 5

### Large-Scale Induction of Hematopoietic Cells

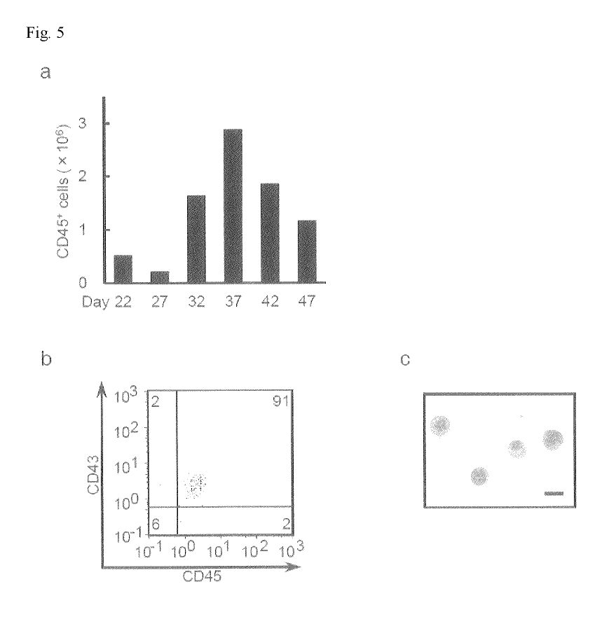

In order to whether or not the culture method using a CS in the present invention can be applied to large-scale suspension culture, an experiment was carried out using a 50-mL flask. Briefly, differentiation of small clusters of PSCs (KhES1) was induced on a plurality of CSs by the method in Example 2, and, on Day 18 after the differentiation, 12 CSs were transferred together into a 50-mL flask (tissue culture T75 flask (Becton-Dickinson)) containing 25 mL of a medium supplemented with a myeloid cytokine cocktail. Thereafter, the CSs were cultured under suspension culture conditions while floating cells were collected every time when medium replacement was carried out. The collection of floating cells was carried out by gently suspending the content of the flask, collecting the medium containing the floating cells, and then subjecting the collected medium to centrifugation. The collection of the floating cells was repeated for two weeks. As a result, 1,000,000 cells were collected from the 12 CSs (FIG. 5a). Most of the collected cells were CD43-, and CD45-positive cells, and they had the same shape as that of immature myeloid cells (FIG. 5b and FIG. 5c). That is, the above results suggest that the differentiation induction system based on CSs can be applied to large-scale induction of hematopoietic cells from pluripotent stem cells.

### INDUSTRIAL APPLICABILITY

The present invention provides a method for stably supplying a large amount of hematopoietic cells. Thus, treatment of various blood diseases is possible.

1. A method for producing mesodermal cells from pluripotent stem cells, comprising culturing pluripotent stem cells in contact with a three-dimensional support to induce mesodermal cells.

2. The method according to claim 1, wherein said mesodermal cells are KDR-, and CD34-positive cells.

3. The method according to claim 1, wherein said three-dimensional support is a collagen sponge.

4. The method according to claim 3, wherein said collagen sponge is a collagen sponge reinforced with polyethylene terephthalate fibers.

5. The method according to claim 1, wherein the contact between said pluripotent stem cells and said three-dimensional support occurs on a surface and/or in the inside of said three-dimensional support.

6. The method according to claim 1, wherein the step of culturing said pluripotent stem cells in contact with said three-dimensional support comprises:
(i) culturing said pluripotent stem cells in a medium containing BMP4; and
(ii) culturing cells obtained in Step (i) in a medium containing VEGF, bFGF, and SCF.

7. The method according to claim 6, wherein the culture periods of steps (i) and (ii) are 1 to 5 days and 0.5 to 3 days, respectively.

8. The method according to claim 7, wherein the culture periods of steps (i) and (ii) are 3 days and 1 day, respectively.

9. The method according to claim 6, wherein preculture is carried out by bringing pluripotent stem cells into contact with the three-dimensional support before step (i).

10. The method according to claim 1, wherein said pluripotent stem cells are human iPS cells.

11. The method according to claim 1, wherein said pluripotent stem cells are small clusters or single cells.

12. The method according to claim 11, wherein said pluripotent stem cells are single cells.

13. A mesodermal cell supported by a three-dimensional support, produced by the method according to claim 1.

14. A graft material comprising the mesodermal cell supported by a three-dimensional support according to claim 13.

15. A culture vessel retaining one or more three-dimensional supports, wherein mesodermal cells are supported by said three-dimensional support(s), and said mesodermal cells are cells produced by the method according to claim 1.

16. A method for producing hematopoietic cells, comprising:
(a) producing mesodermal cells in contact with a three-dimensional support by the method according to claim 1; and
(b) culturing the obtained mesodermal cells retained by the three-dimensional support in a culture vessel to induce hematopoietic cells.

17. The method according to claim 16, wherein said hematopoietic cells are myeloid cells, monocytic cells, or erythroid cells.

18. The method according to claim 16, wherein said hematopoietic cells are myeloid cells.

19. The method according to claim 16, wherein said hematopoietic cells are monocytic cells.

20. The method according to claim 16, wherein said hematopoietic cells are erythroid cells.

21. The method according to claim 18, wherein the step (b) comprises culturing the mesodermal cells retained by the three-dimensional support in a medium containing SCF, IL-3, Flt3L, and thrombopoietin (TPO).

22. The method according to claim 19, wherein the step (b) comprises:
(i) culturing the mesodermal cells retained by the three-dimensional support in a medium containing SCF, IL-3, Flt3L, and TPO;
(ii) culturing cells obtained in the step (i) in a medium containing SCF, IL-3, Flt3L, TPO, and M-CSF; and
(iii) culturing cells obtained in the step (ii) in a medium containing Flt3L, M-CSF, and GM-CSF.

23. The method according to claim 20, wherein the step (b) comprises culturing the mesodermal cells retained by the three-dimensional support in a medium containing erythropoietin (EPO) and SCF.

24. The method according to claim 21, wherein the culture period of the step (b) is 16 to 41 days.

25. The method according to claim 24, wherein the culture period of the step (b) is 31 days.

26. The method according to claim 22, wherein the culture periods of the steps (i), (ii), and (iii) are 2 to 5 days, 2 to 5 days, and 8 to 33 days, respectively.

27. The method according to claim 26, wherein the culture periods of the steps (i), (ii), and (iii) are 3 days, 3 days, and 23 days, respectively.

28. A therapeutic agent for blood diseases, comprising a hematopoietic cell produced by the method according to claim 16.

29. The method according to claim 23, wherein the culture period of the step (b) is 16 to 41 days.

30. The method according to claim 29, wherein the culture period of the step (b) is 31 days.
